SEQUENCE LISTING (50 sequences)

SEQ ID NO: 1 - protein, 140 aa, synthetic construct
  VARIANT 14: note = S, V, W, A, C, F, G, I, L, M, P
  VARIANT 66: note = N, Q, S, T, Y
  VARIANT 92: note = S, V, W, A, C, F, G, I, L, M, P
  VARIANT 93: note = S, V, W, A, C, F, G, I, L, M, P
  VARIANT 107: note = T, V, W, A, C, F, G, I, L, M, P
  VARIANT 115: note = S, V, W, A, C, F, G, I, L, M, P
  VARIANT 118: note = G, I, L, M, P, V, W, A, C, F
  VARIANT 119: note = F, G, I, L, M, P, V, W, A, C
IQNPDPAVYQLRDXKSSDKSVCLFTDFDSQTNVSQSKDSDVYITDKTVLDMRSMDFKSNSAVAWSXKSDFACANAFNNSIIPEDTFFPSPEXXCDVKLVEKSFETDXNLNFQNLXVIXXRILLLKVAGFNLLMTLRLWSS

SEQ ID NO: 2 - protein, 179 aa, synthetic construct
  VARIANT 4: note = K, N
  VARIANT 37: note = Y, F
  VARIANT 136: note = E, V
  VARIANT 177: note = S, F
  VARIANT 178: note = R or deleted
  VARIANT 179: note = G or deleted
  VARIANT 5: note = N, R, D, E, H, K
  VARIANT 18: note = E, H, K, R, D
  VARIANT 22: note = S, V, W, A, C, F, G, I, L, M, P
  VARIANT 34: note = T, D, E, H, K, N, R
  VARIANT 157: note = T, V, W, A, C, F, G, I, L, M, P
  VARIANT 164: note = S, V, W, A, C, F, G, I, L, M, P
EDLXXVFPPEVAVFEPSXAEIXHTQKATLVCLAXGFXPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSXSYQQGVLSATILYEILLGKAXLYAVLVXALVLMAMVKRKDXXX

SEQ ID NO: 3 - protein, 9 aa, synthetic construct
VLDGLDVLL

SEQ ID NO: 4 - protein, 6 aa, synthetic construct
VSGLRG

SEQ ID NO: 5 - protein, 7 aa, synthetic construct
LYSAGEE

SEQ ID NO: 6 - protein, 15 aa, synthetic construct
CAVHSTAQAGTALIF

SEQ ID NO: 7 - protein, 5 aa, synthetic construct
SGDLS

SEQ ID NO: 8 - protein, 6 aa, synthetic construct
YYNGEE

SEQ ID NO: 9 - protein, 16 aa, synthetic construct
CASSTHRGQTNYGYTF

SEQ ID NO: 10 - protein, 134 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVHSTAQAGTALIFGKGTTLSVSSN

SEQ ID NO: 11 - protein, 134 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSTHRGQTNYGYTFGSGTRLTVV

SEQ ID NO: 12 - protein, 140 aa, synthetic construct
IQNPDPAVYQLRDSKSSDKSVCLFTDFDSQTNVSQSKDSDVYITDKTVLDMRSMDFKSNSAVAWSSKSDFACANAFNNSIIPEDTFFPSPEMACDVKLVEKSFETDTNLNFQNLLVIWLRILLLKVAGFNLLMTLRLWSS

SEQ ID NO: 13 - protein, 179 aa, synthetic construct
EDLKHVFPPEVAVFEPSDAEIWHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 14 - protein, 9 aa, synthetic construct
SLLQHLIGL

SEQ ID NO: 15 - protein, 177 aa, synthetic construct
  VARIANT 5: note = N, R, D, E, H, K
  VARIANT 18: note = E, H, K, R, D
  VARIANT 22: note = S, V, W, A, C, F, G, I, L, M, P
  VARIANT 34: note = T, D, E, H, K, N, R
  VARIANT 157: note = T, V, W, A, C, F, G, I, L, M, P
  VARIANT 164: note = S, V, W, A, C, F, G, I, L, M, P
EDLNXVFPPEVAVFEPSXAEIXHTQKATLVCLAXGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKAXLYAVLVXALVLMAMVKRKDF

SEQ ID NO: 16 - protein, 179 aa, synthetic construct
  VARIANT 5: note = N, R, D, E, H, K
  VARIANT 18: note = E, H, K, R, D
  VARIANT 22: note = S, V, W, A, C, F, G, I, L, M, P
  VARIANT 34: note = T, D, E, H, K, N, R
  VARIANT 157: note = T, V, W, A, C, F, G, I, L, M, P
  VARIANT 164: note = S, V, W, A, C, F, G, I, L, M, P
EDLKXVFPPKVAVFEPSXAEIXHTQKATLVCLAXGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKAXLYAVLVXALVLMAMVKRKDSRG

SEQ ID NO: 17 - protein, 140 aa, synthetic construct
IQNPDPAVYQLRDSKSSDKSVCLFTDFDSQTNVSQSKDSDVYITDKTVLDMRSMDFKSNSAVAWSTKSDFACANAFNNSIIPEDTFFPSPEVVCDVKLVEKSFETDTNLNFQNLVVIGARILLLKVAGFNLLMTLRLWSS

SEQ ID NO: 18 - protein, 179 aa, synthetic construct
EDLKNVFPPEVAVFEPSRAEISHTQKATLVCLARGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 19 - protein, 140 aa, synthetic construct
IQNPDPAVYQLRDWKSSDKSVCLFTDFDSQTNVSQSKDSDVYITDKTVLDMRSMDFKSNSAVAWSQKSDFACANAFNNSIIPEDTFFPSPEMPCDVKLVEKSFETDWNLNFQNLLVIGARILLLKVAGFNLLMTLRLWSS

SEQ ID NO: 20 - protein, 179 aa, synthetic construct
EDLKNVFPPEVAVFEPSRAEIVHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKACLYAVLVSALVLMAMVKRKDSRG

SEQ ID NO: 21 - protein, 140 aa, synthetic construct
IQNPDPAVYQLRDSKSSDKSVCLFTDFDSQTNVSQSKDSDVYITDKTVLDMRSMDFKSNSAVAWSTKSDFACANAFNNSIIPEDTFFPSPEGWCDVKLVEKSFETDVNLNFQNLSVIFMRILLLKVAGFNLLMTLRLWSS

SEQ ID NO: 22 - protein, 179 aa, synthetic construct
EDLKRVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 23 - protein, 140 aa, synthetic construct
IQNPDPAVYQLRDIKSSDKSVCLFTDFDSQTNVSQSKDSDVYITDKTVLDMRSMDFKSNSAVAWSYKSDFACANAFNNSIIPEDTFFPSPEIFCDVKLVEKSFETDANLNFQNLSVIALRILLLKVAGFNLLMTLRLWSS

SEQ ID NO: 24 - protein, 179 aa, synthetic construct
EDLKRVFPPEVAVFEPSDAEIGHTQKATLVCLANGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVCALVLMAMVKRKDSRG

SEQ ID NO: 25 - protein, 140 aa, synthetic construct
IQNPDPAVYQLRDMKSSDKSVCLFTDFDSQTNVSQSKDSDVYITDKTVLDMRSMDFKSNSAVAWSTKSDFACANAFNNSIIPEDTFFPSPESGCDVKLVEKSFETDPNLNFQNLIVIFLRILLLKVAGFNLLMTLRLWSS

SEQ ID NO: 26 - protein, 179 aa, synthetic construct
EDLKEVFPPEVAVFEPSDAEIMHTQKATLVCLAEGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKALLYAVLVSALVLMAMVKRKDSRG

SEQ ID NO: 27 - protein, 140 aa, synthetic construct
IQNPDPAVYQLRDAKSSDKSVCLFTDFDSQTNVSQSKDSDVYITDKTVLDMRSMDFKSNSAVAWSTKSDFACANAFNNSIIPEDTFFPSPEIICDVKLVEKSFETDPNLNFQNLVVILLRILLLKVAGFNLLMTLRLWSS

SEQ ID NO: 28 - protein, 179 aa, synthetic construct
EDLKHVFPPEVAVFEPSDAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 29 - protein, 140 aa, synthetic construct
IQNPDPAVYQLRDMKSSDKSVCLFTDFDSQTNVSQSKDSDVYITDKTVLDMRSMDFKSNSAVAWSTKSDFACANAFNNSIIPEDTFFPSPEPLCDVKLVEKSFETDPNLNFQNLAVIAMRILLLKVAGFNLLMTLRLWSS

SEQ ID NO: 30 - protein, 179 aa, synthetic construct
EDLKEVFPPEVAVFEPSEAEIAHTQKATLVCLANGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 31 - protein, 140 aa, synthetic construct
IQNPDPAVYQLRDMKSSDKSVCLFTDFDSQTNVSQSKDSDVYITDKTVLDMRSMDFKSNSAVAWSNKSDFACANAFNNSIIPEDTFFPSPEWFCDVKLVEKSFETDTNLNFQNLCVIVIRILLLKVAGFNLLMTLRLWSS

SEQ ID NO: 32 - protein, 179 aa, synthetic construct
EDLKNVFPPEVAVFEPSDAEIIHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 33 - protein, 9 aa, synthetic construct
SLLMWITQC

SEQ ID NO: 34 - protein, 6 aa, synthetic construct
DRGSQS

SEQ ID NO: 35 - protein, 6 aa, synthetic construct
IYSNGD

SEQ ID NO: 36 - protein, 9 aa, synthetic construct
CAADDKIIF

SEQ ID NO: 37 - protein, 5 aa, synthetic construct
SGHDY

SEQ ID NO: 38 - protein, 6 aa, synthetic construct
FNNNVP

SEQ ID NO: 39 - protein, 10 aa, synthetic construct
CASSRGGSGY

SEQ ID NO: 40 - protein, 129 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAADDKIIFGKGTRLHILPN

SEQ ID NO: 41 - protein, 132 aa, synthetic construct
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSRGGSGYTFGSGTRLTVVE

SEQ ID NO: 42 - protein, 269 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAADDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 43 - protein, 311 aa, synthetic construct
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSRGGSGYTFGSGTRLTVVEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSG

SEQ ID NO: 44 - protein, 310 aa, synthetic construct
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSRGGSGYTFGSGTRLTVVEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 45 - protein, 308 aa, synthetic construct
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSRGGSGYTFGSGTRLTVVEDLNHVFPPEVAVFEPSDAEIWHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 46 - protein, 310 aa, synthetic construct
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSRGGSGYTFGSGTRLTVVEDLKHVFPPEVAVFEPSDAEIWHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 47 - protein, 269 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAADDKIIFGKGTRLHILPNIQNPDPAVYQLRDSKSSDKSVCLFTDFDSQTNVSQSKDSDVYITDKTVLDMRSMDFKSNSAVAWSSKSDFACANAFNNSIIPEDTFFPSPEMACDVKLVEKSFETDTNLNFQNLLVIWLRILLLKVAGFNLLMTLRLWSS

SEQ ID NO: 48 - protein, 269 aa, synthetic construct; identical to SEQ ID NO: 42

SEQ ID NO: 49 - protein, 308 aa, synthetic construct
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSRGGSGYTFGSGTRLTVVEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 50 - protein, 310 aa, synthetic construct
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSRGGSGYTFGSGTRLTVVEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGTTSASYHQGVLSATILYEILLGKATLYAVLVSALVLMAMVKRKDSRG

(19) United States
(12) Patent Application Publication
(10) Pub. No.: US 2025/0197848 A1
(43) Pub. Date: Jun. 19, 2025

(54) TCR CONSTANT REGION PAIRING LIBRARY

(71) Applicant: MEDIGENE IMMUNO THERAPIES GMBH, Planegg-Martinsried (DE)

(72) Inventors: Slavoljub MILOSEVIC, München (DE); Adriana TURQUETI NEVES, Olching (DE); Andreas ACS, Nürnberg (DE); Justyna OGONEK, München (DE)

(21) Appl. No.: 18/846,736

(22) PCT Filed: Mar. 16, 2023
(86) PCT No.: PCT/EP2023/056739

§ 371 Date: Sep. 13, 2024

(30) Foreign Application Priority Data
Mar. 16, 2022 (EP) 22162441.4

(51) Int. Cl. C12N 15/10; C07K 14/725; C12N 15/86; C40B 40/08

(52) U.S. Cl. CPC C12N 15/1086; C07K 14/7051; C12N 15/1065; C12N 15/86; C40B 40/08; C12N 2740/10043

(57) ABSTRACT

The present invention relates to a library of synthetic polynucleotides encoding TCR alpha chain constant regions and TCR beta chain constant regions for enhanced paring of recombinant TCR alpha and TCR beta chains and/or enhanced surface expression of recombinant TCRs. The invention further refers to corresponding vector libraries, TCR libraries, cell libraries and methods for isolating TCRs with enhanced TCR alpha and TCR beta chain pairing using said libraries and TCRs isolated from said libraries.

### FIELD OF THE INVENTION

The present invention relates to a library of synthetic polynucleotides encoding TCR alpha chain constant regions and TCR beta chain constant regions for enhanced pairing of recombinant TCR alpha and TCR beta chains and/or enhanced surface expression of recombinant TCRs. The invention further refers to corresponding vector libraries, TCR libraries, cell libraries and methods for isolating TCRs with enhanced TCR alpha and TCR beta chain pairing using said libraries and TCRs isolated from said libraries.

### BACKGROUND OF THE INVENTION

T lymphocytes are part of the adaptive immune response and originate from hematopoietic stem cells located in the bone marrow. T lymphocytes express a unique antigen binding receptor on their membrane, the T cell receptor (TCR), which recognizes antigens in association with major histocompatibility complex (MHC) molecules.

With their central role in the immune system, T cells typically provide protection from pathogens or malignant cells. Each T cell expresses a single form of a T cell receptor, a structure which is used by the T cell to recognize infected or altered cells.

The concept of immunotherapy is based on the specificity of the adaptive immune response for the recognition and elimination of pathogens as well as tumor cells. The aim of a successful immunotherapy is the manipulation or reprogramming of the patient's immune response in order to specifically target pathogen-infected cells or tumor cells for destruction by the immune system.

Therapeutic approaches used to reprogram the immune system in the treatment of infectious diseases and cancer include active immunotherapy comprising the use of vaccination strategies, including dendritic cell (DC) vaccines, as well as passive immunotherapy comprising the application of specific antibodies or genetically engineered lymphocytes or the adoptive transfer of T cells specifically recognizing target antigens displayed by pathogen-infected cells or cancers.

The principle of adoptive T cell transfer is based on the ex vivo expansion of autologous or allogeneic target-specific T lymphocytes and the subsequent re-infusion into patients. Cancer regression in patients suffering from metastatic melanoma has been observed after the transfer of ex vivo expanded autologous tumor-infiltrating lymphocytes (TILs). The drawback of this therapeutic approach is the requirement for pre-existing tumor-reactive cells that need to be isolated from every individual patient as well as the difficult detection of TILs for cancers other than melanoma. Therefore, other methods were developed that focus on the genetic modification of T cells isolated from patients. These genetically engineered T cells can for example be created by transduction of autologous T cells with the α and β chains of target-specific TCRs, i.e. with recombinant TCRs. Likewise, there have also been successful treatments of life-threatening infections with adoptive transfer of virus-specific T cells, for example to combat Epstein-Barr-Virus- and Cytomegalovirus-driven infections in immune-compromised individuals. This is a pathway that could also be pursued for treatment of COVID-19 patients in dire situations.

An exemplary approach, as developed by Wilde et al. in 2009 and described in WO 2007/017201, allows the isolation of allo-restricted peptide-specific T cells using autologous DCs co-transfected with RNA species encoding both the target antigen and a selected allogeneic MHC molecule. By co-culturing autologous T cells with DCs presenting self-peptide/allo-MHC complexes, high-avidity T cells that recognize self-antigens can be obtained (Wilde et al., 2009, Dendritic cells pulsed with RNA encoding allogeneic MHC and antigen induce T cells with superior antitumor activity and higher TCR functional avidity. Blood, 114 (10), 2131-9). In another exemplary approach, T cells and their corresponding TCRs can be obtained by culturing autologous T cells and autologous DCs expressing the human leukocyte antigens (HLAs) of choice and loaded with foreign antigens, such as those expressed by pathogenic viruses in infected cells. Because T cell cultivation and expansion of individual T cell clones is laborious and requires repeated rounds of re-stimulation, it is an advantage to rapidly acquire the sequences of the TCRs at an early time point in order to allow their rapid characterization by introducing them into recipient peripheral blood lymphocyte-containing T cells. This allows the characterization of the TCRs regarding antigen specificity, peptide/MHC-avidity and functionality before they are selected for further use in therapeutic applications in patients.

However both safety and efficacy of TCR-T immunotherapy need further to be improved. For efficient immunotherapy strong surface expression of the TCR is vital. A prerequisite for high expression of the TCR on the cell surface is the correct pairing of the TCR alpha and TCR beta chains. In particular, if endogenous TCR chains are present, mispairing of the recombinant TCR chains with the endogenous TCR chains must be avoided.

Hence there is a need for methods to improve the preferential pairing of the recombinant TCR alpha and TCR beta chains and improved pairing with the CD3 complex.

### OBJECTIVES AND SUMMARY OF THE INVENTION

The present invention provides libraries and methods for improving the pairing of the recombinant TCR alpha and TCR beta chains. There by the surface expression of the transgenic TCR chains can be increased. The inventors could show that surprisingly these strategies lead to TCRs with increased functional avidity.

In particular, in TCR-T immunotherapy when recombinant TCRs are expressed in T cells carrying the endogenous TCR, the mispairing of recombinant TCR chains with endogenous TCR chains can be reduced.

The inventors identified specific positions in the constant regions of the TCR alpha and TCR beta chain that impact binding of both chains and collated a library of tailored amino acid substitutions in the constant regions of the TCR alpha and TCR beta chains, which can be introduced to potentially elicit improved binding between the chains. The inventors found that the improved binding by the modified chains resulted in enhanced TCR surface expression and function leading to TCR-Ts expressing such TCRs with improved in vitro efficacy in target cell recognition.

Thus, a first aspect of the invention relates to a library of synthetic polynucleotides encoding TCR alpha chain constant regions and TCR beta chain constant regions, wherein the library comprises:
polynucleotides encoding TCR alpha chain constant regions which are selected from the group of TCR alpha constant regions which are at least about 80% identical to the amino acid sequence as set out in SEQ ID NO: 1;
polynucleotides encoding TCR beta chain constant regions which are selected from the group of TCR beta constant regions which are at least about 80% identical to the amino acid sequence as set out in SEQ ID NO: 2.

Accordingly, the invention also relates to a library of synthetic polynucleotides encoding TCR alpha chains and TCR beta chains, comprising
Polynucleotides encoding TCR alpha chains comprising constant regions which are selected from the group of TCR alpha constant regions which are at least about 80% identical to the amino acid sequence as set out in SEQ ID NO: 1;
Polynucleotides encoding TCR beta chains comprising constant regions which are selected from the group of TCR beta constant regions which are at least about 80% identical to the amino acid sequence as set out in SEQ ID NO: 15; or Polynucleotides encoding TCR beta chains comprising constant regions which are selected from the group of TCR beta constant regions which are at least about 80% identical to the amino acid sequence as set out in SEQ ID NO: 16.

In one embodiment the library comprises polynucleotides encoding a TCR alpha chain and a TCR beta chain, each polynucleotide comprising:
Polynucleotide encoding TCR alpha chains as defined herein; and
Polynucleotide encoding TCR beta chains as defined herein;
Optionally a selection marker
Optionally one or more internal ribosomal entry sites or self-cleaving peptides of the 2A family.

Typically, the library comprises at least 1×10⁵, preferably 1×10⁸, more preferably 1×10¹⁰, even more preferably 1×10¹¹, such as 2.25×10¹³ unique molecules.

Also encompassed is a library of vectors comprising the polynucleotide library as defined herein. Further encompassed is a library of cells, comprising said library of vectors. Moreover, also a library of cells expressing the polynucleotides as defined herein is encompassed. A further aspect relates to a library of polypeptides encoded by the library of synthetic polynucleotides as defined herein.

Another aspect refers to the use of the library as described herein for identifying a TCR receptor with enhanced TCR alpha and TCR beta chain pairing.

A further aspect refers to a method for isolating a TCR with enhanced TCR alpha and beta chain pairing, comprising the steps:
Co-culturing the library of cells according to claims 9 to 11 with antigen presenting cells presenting the HLA bound form of the target antigen of the TCR;
and at least one of the steps:
Selecting cells showing elevated expression levels of the heterologous TCR compared to wild type
Selecting cells showing elevated activation levels compared to wild type.

Accordingly, also a TCR isolated from the TCR library as described herein is encompassed.

Another aspect refers to a library of TCR alpha chains and TCR beta chains, comprising
TCR alpha chains comprising constant regions which are selected from the group of TCR alpha constant regions which are at least about 80% identical to the amino acid sequence as set out in SEQ ID NO: 1;
TCR beta chains comprising constant regions which are selected from the group of TCR beta constant regions which are at least about 80% identical to the amino acid sequence as set out in SEQ ID NO: 2.

Also encompassed is s method of preparing a library of synthetic polynucleotides encoding a plurality of TCR alpha chain constant regions and TCR beta chain constant regions, the method comprising:
Providing sequences of polynucleotides
   (i) encoding TCR alpha chain constant regions and TCR beta chain constant regions as defined in herein; or
   (ii) encoding the TCR alpha chain and TCR beta chain as defined in herein;
Assembling the synthetic polynucleotide sequences to produce a library of synthetic polynucleotides.

A further aspect refers to a kit comprising one or more of the libraries described herein.

Another aspect of the invention refers to nucleotide sequences of the synthetic polynucleotides defined herein in computer readable format. Accordingly, the invention also encompasses the amino acid sequences as defined herein in computer readable format.

Further embodiments refer to libraries and methods for improving the pairing of the recombinant TCR alpha and TCR beta chains of TCRs targeting NY-ESO-1.

Accordingly, some embodiments relate to a library of synthetic polynucleotides encoding TCR alpha chains and TCR beta chains of a TCR capable of binding to a NY-ESO-1 peptide having the sequence SLLMWITQC (SEQ ID NO: 33), comprising
Polynucleotides encoding TCR alpha chains comprising:
a) TCR alpha chain constant regions which are selected from the group of TCR alpha constant regions which are at least about 80% identical to the amino acid sequence as set out in SEQ ID NO: 1;
wherein
the amino acid at position 14 is selected from the group consisting of S, V, W, A, C, F, G, I, L, M and P;
the amino acid at position 66 is selected from the group consisting of N, Q, S, T and Y;
the amino acid at position 92 is selected from the group consisting of S, V, W, A C, F, G, I, L, M and P;
the amino acid at position 93 is selected from the group consisting of S, V, W, A C, F, G, I, L, M and P;
the amino acid at position 107 is selected from the group consisting of T, V, W, A, C, F, G, I, L, M and P;
the amino acid at position115 is selected from the group consisting of S, V, W, A, C, F, G, I, L, M and P;
the amino acid at position 118 is selected from the group consisting of G, I, L, M, P, V, W, A, C and F; and
the amino acid at position 119 is selected from the group consisting of F, G, I, L, M, P, V, W, A and C;
b) TCR alpha chain variable region comprising a CDR1 having the amino acid sequence of SEQ ID NO: 34, a CDR2 having the amino acid sequence of SEQ ID NO: 35 and a CDR3 having the amino acid sequence of SEQ ID NO: 36;
Polynucleotides encoding TCR beta chains comprising
a) TCR beta chain constant regions which are selected from the group of TCR beta constant regions which are at least about 80% identical to the amino acid sequence as set out in SEQ ID NO: 2
   wherein
   the amino acid at position at position 4 is selected from the group consisting of K, N;
   the amino acid at position at position 5 is selected from the group consisting of N, R, D, E, H and K;
   the amino acid at position at position 18 is selected from the group consisting of E, H, K, R and D;
   the amino acid at position at position 22 is selected from the group consisting of S, V, W, A, C, F, G, I, L, M and P;
   the amino acid at position at position 34 is selected from the group consisting of T, D, E, H, K, N and R;
   the amino acid at position 37 is selected from the group consisting of Y, F;
   the amino acid at position 136 is selected from the group consisting of E, V;
   the amino acid at position 157 is selected from the group consisting of T, V, W, A, C, F, G, I, L, M and P;
   the amino acid at position 164 is selected from the group consisting of S, V, W, A, C, F, G, I, L, M and P;
   the amino acid at position 177 is selected from the group consisting of S, F;
   the amino acid at position 178 is R or deleted; and
   the amino acid at position 179 is G or deleted.
b) TCR beta chain variable region comprising a CDR1 having the amino acid sequence of SEQ ID NO: 37, a CDR2 having the amino acid sequence of SEQ ID NO: 38 and a CDR3 having the amino acid sequence of SEQ ID NO: 39.

Some embodiments relate to a library of synthetic polynucleotides encoding TCR alpha chains and TCR beta chains of a TCR capable of binding to a NY-ESO-1 peptide having the sequence SLLMWITQC (SEQ ID NO: 33), comprising
Polynucleotides encoding TCR alpha chains comprising:
a) TCR alpha chain constant regions which are selected from the group of TCR alpha constant regions which are at least about 80% identical to the amino acid sequence as set out in SEQ ID NO: 1:
wherein
the amino acid at position 14 is selected from the group consisting of S, V, W, A, C, F, G, I, L, M and P;
the amino acid at position 66 is selected from the group consisting of N, Q, S, T and Y;
the amino acid at position 92 is selected from the group consisting of S, V, W, A C, F, G, I, L, M and P;
the amino acid at position 93 is selected from the group consisting of S, V, W, A C, F, G, I, L, M and P;
the amino acid at position 107 is selected from the group consisting of T, V, W, A, C, F, G, I, L, M and P;
the amino acid at position115 is selected from the group consisting of S, V, W, A, C, F, G, I, L, M and P;
the amino acid at position 118 is selected from the group consisting of G, I, L, M, P, V, W, A, C and F; and
the amino acid at position 119 is selected from the group consisting of F, G, I, L, M, P, V, W, A and C;
b) TCR alpha chain variable region comprising a CDR1 having the amino acid sequence of SEQ ID NO: 34, a CDR2 having the amino acid sequence of SEQ ID NO: 35 and a CDR3 having the amino acid sequence of SEQ ID NO: 36;
Polynucleotides encoding TCR beta chains comprising
a) TCR beta chain constant regions which are selected from the group of TCR beta constant regions which are at least about 80% identical to the amino acid sequence as set out in SEQ ID NO: 15
wherein
the amino acid at position 5 is selected from the group consisting of N, R, D, E, H and K;
the amino acid at position 18 is selected from the group consisting of E, H, K, R and D;
the amino acid at position 22 is selected from the group consisting of S, V, W, A, C, F, G, I, L, M and P;
the amino acid at position 34 is selected from the group consisting of T, D, E, H, K, N and R;
the amino acid at position 157 is selected from the group consisting of T, V, W, A, C, F, G, I, L, M and P; and
   the amino acid at position 164 is selected from the group consisting of S, V, W, A, C, F, G, I, L, M and P; or
   TCR beta chain constant regions which are selected from the group of TCR beta constant regions which are at least about 80% identical to the amino acid sequence in SEQ ID NO: 16;
wherein
the amino acid at position 5 is selected from the group consisting of N, R, D, E, H and K;
the amino acid at position 18 is selected from the group consisting of E, H, K, R and D;
the amino acid at position 22 is selected from the group consisting of S, V, W, A, C, F, G, I, L, M and P;
the amino acid at position 34 is selected from the group consisting of T, D, E, H, K, N and R;
the amino acid at position 157 is selected from the group consisting of T, V, W, A, C, F, G, I, L, M and P; and
the amino acid at position 164 is selected from the group consisting of S, V, W, A, C, F, G, I, L, M and P;
b) TCR beta chain variable region comprising a CDR1 having the amino acid sequence of SEQ ID NO: 37, a CDR2 having the amino acid sequence of SEQ ID NO: 38 and a CDR3 having the amino acid sequence of SEQ ID NO: 39.

### FIGURE LEGENDS

### Figure Legends

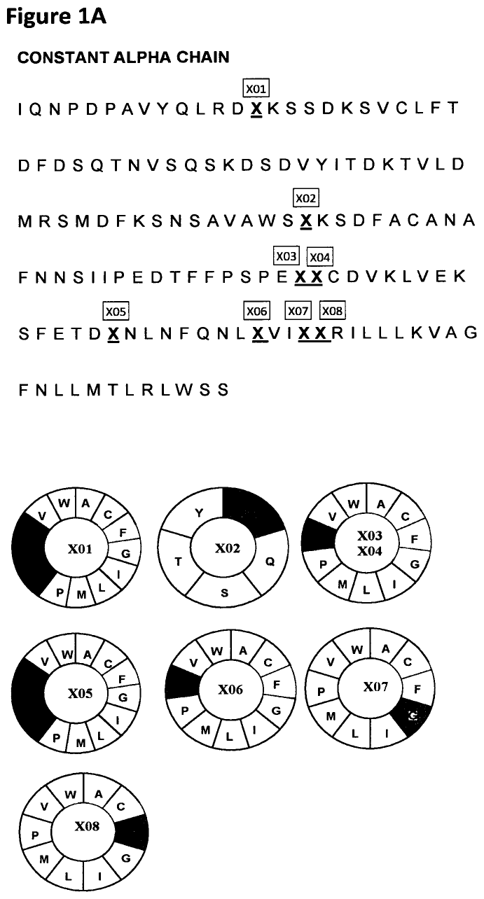

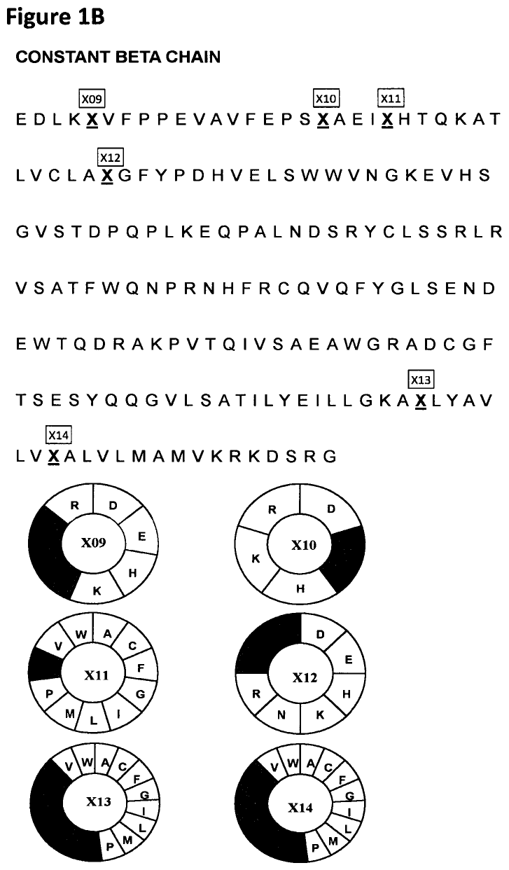

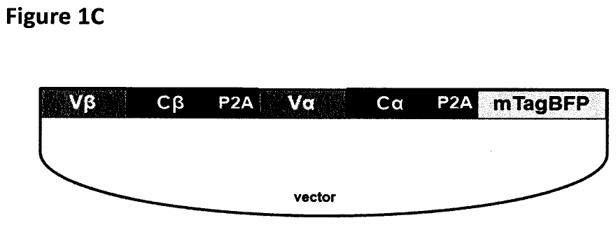

FIG. 1 FIGS. 1A and 1B show the amino acid sequence of the alpha chain constant region as defined in SEQ ID NO: 1 and the beta chain constant region of the TCR constant region as defined in SEQ ID NO: 2 enhancement library, respectively. The introduced mutations are labelled from X01-X08 in the alpha chain (FIG. 1A) and from X09-X14 in the beta chain (FIG. 1B). Donut charts show the distribution at which each amino acid appears in that position. Wild type version of amino acid sequence is highlighted in grey. FIG. 1C visualizes the schematic map of library constructs.

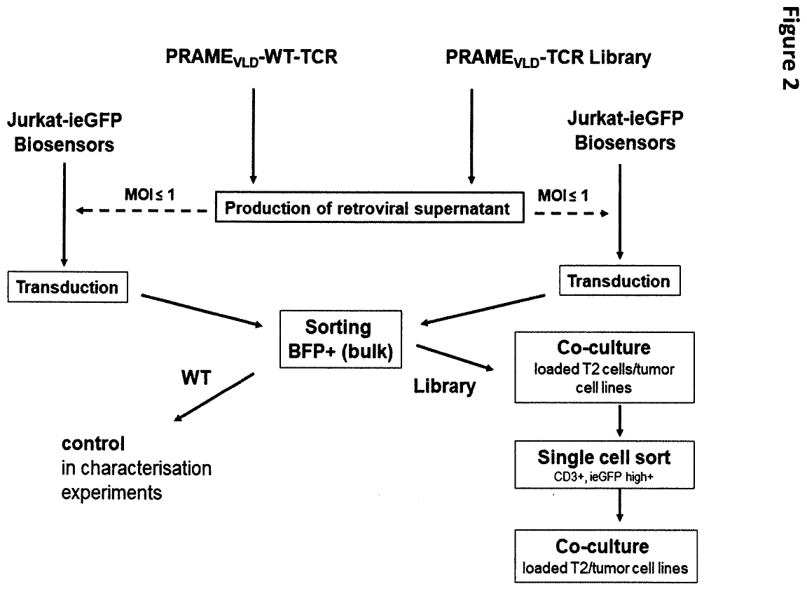

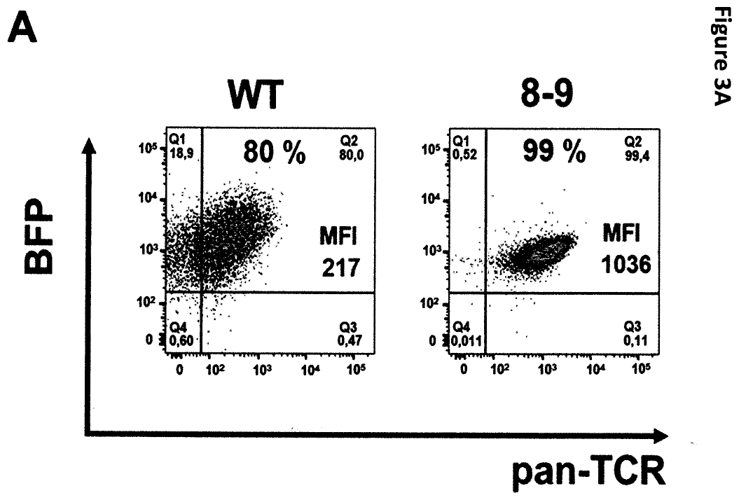

FIG. 2 shows a schematic overview of the screening procedure, which was used to identify a promising TCR clone derived from the library.

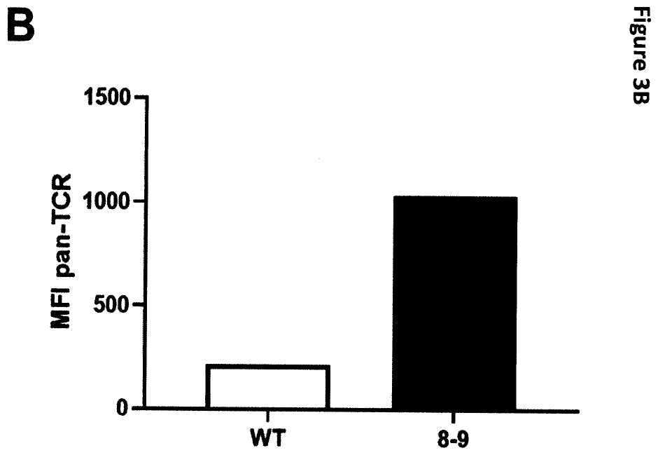

FIG. 3 Comparison of TCR expression on the surface of the promising Jurkat-ieGFP Biosensor library clone (8-9) and Jurkat-ieGFP Biosensor carrying PRAME_{VLD}-WT-TCR (WT). Cells were stained with an antibody recognizing human monomorphic determinant of the α/β chain of the TCR (anti-panTCR-Allophycocyanin, APC) and analyzed with flow cytometry. (A) Dot plots presenting co-expression of blue fluorescent protein (BFP-transduction marker) and TCR (% of BFP⁺TCR⁺ cells is shown). (B) Graph presenting the level of TCR expression as a median fluorescence intensity (MFI) of pan-TCR-APC signal.

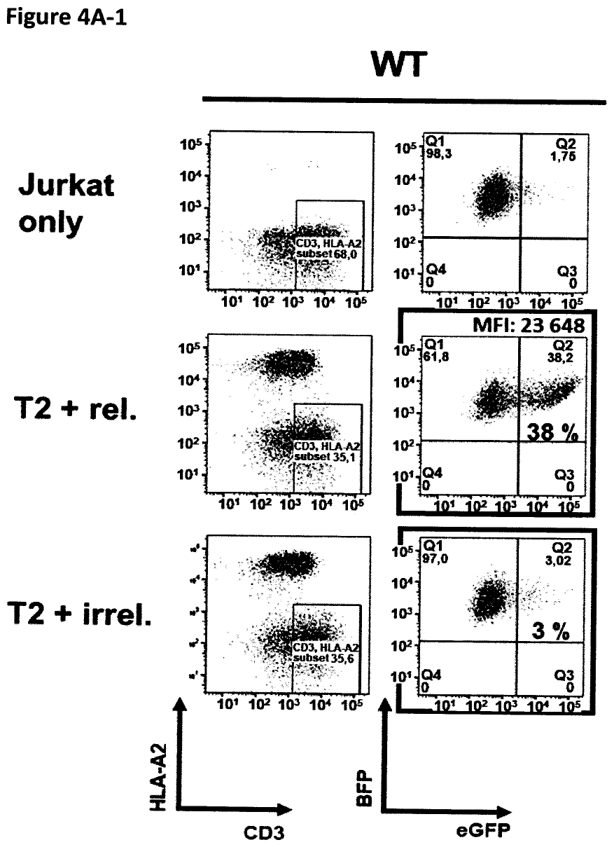

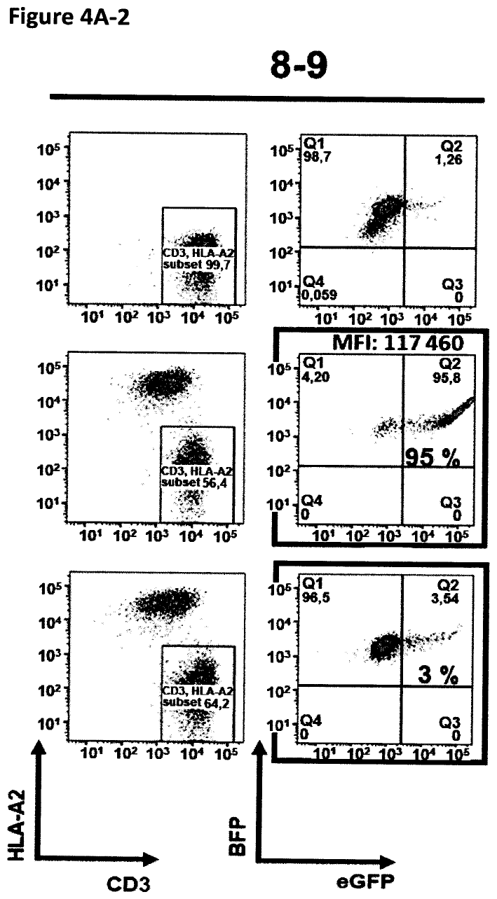

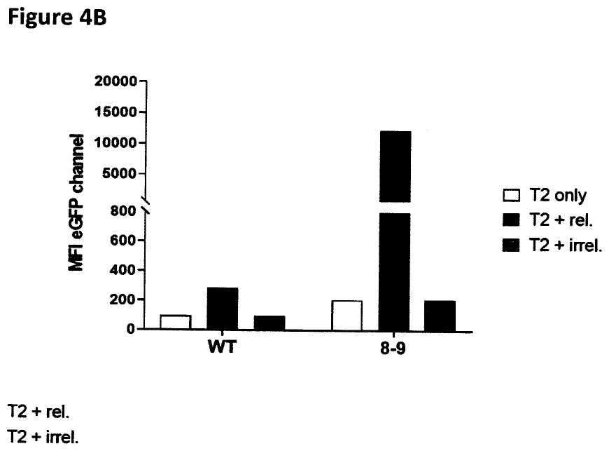

FIG. 4 Testing TCR specificity of the selected clone. 8-9 clone and WT were co-cultured with T2 cells loaded with either relevant (rel.) VLDGLDVLL peptide (T2+rel.), irrelevant control (irrel.) peptide SLLQHLIGL (T2+irrel.) or left alone (Jurkat only). (A) After 24h of incubation, cells were stained with anti-HLA-A2-APC and anti-CD3-phycoerythrin-cyanine7 (PE-Cy7) antibody and acquired at flow cytometry. Percentages of eGFP+Jurkat cells and eGFP MFI for conditions with rel. and irrel. peptide are marked in bold (B) In a separate experiment, cells without additional staining were analyzed. MFI of eGFP channel was plotted.

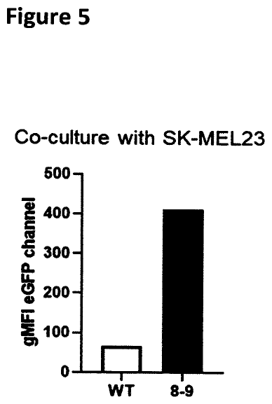

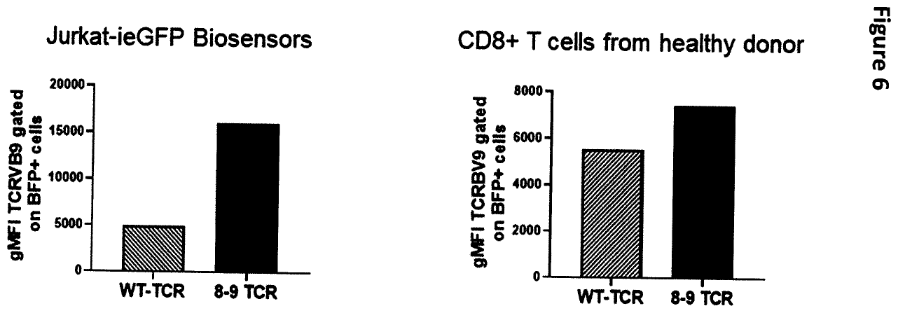

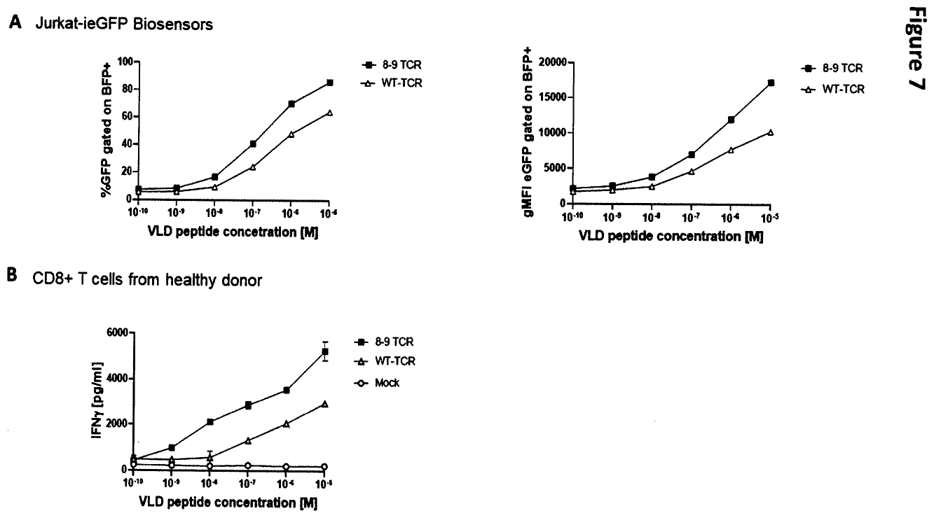

FIG. 5 Reactivity of the selected clone to the naturally processed and presented peptide. 8-9 and WT were co-cultured with tumor cell line (SK-MEL23-human melanoma) in the 1:1 ratio. After 24h, eGFP signal on Jurkat-ieGFP Biosensors was measured with flow cytometry. Graphs show geometric mean fluorescence intensity (gMFI) of eGFP channel.

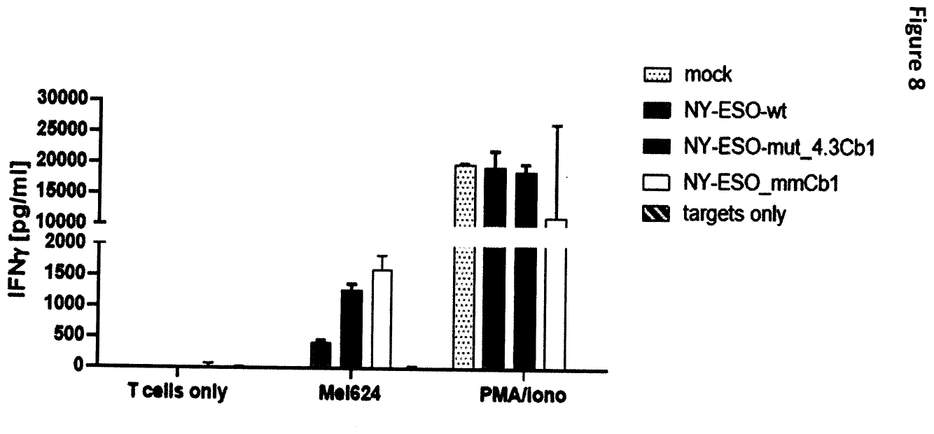

FIG. 6 TCR expression of reconstituted 8-9 TCR and WT-TCR on the surface of Jurkat-ieGFP Biosensor and CD8+ T cells from a healthy donor. Cells were stained with anti-CD3-APC antibody, an antibody recognizing variable B1 chain of the TCR (anti-TCRBV9-PE), and live/dead marker 7-Amino-Actinomycin D (7AAD) and acquired at flow cytometer. Graph presenting the level of TCR expression as a gMFI of TCRBV9-PE signal on BFP⁺ cells is shown.

FIG. 7 Comparison of functional avidity between reconstituted 8-9 and WT-TCRs. (A) 8-9 TCR or WT-TCR-transduced Jurkat-ieGFP Biosensor cells (effectors) were co-cultured with T2 cells (targets) loaded with titrated concentrations of VLD peptide ranging from 10⁻¹⁰ to 10⁻⁵ M. Effector to target ratio was 2:1 (50000:25000 cells). After 20h of co-culture, cells were collected and stained with anti-HLA-A2-APC antibody to visualize target cells. Percentage of eGFP positive cells and gMFI of eGFP signal on the HLA-A2⁻BFP⁺ cells (transduced effector cells) was measured with flow cytometry and plotted on the graph at different co-culture conditions. (B) 8-9 TCR-, WT-TCR- or mock-transduced CD8⁺ T cells from a healthy donor were co-cultured with T2 cells loaded with titrated VLD peptide concentrations ranging from 10⁻¹⁰ to 10⁻⁵ M. After 24 h, supernatants were collected and IFNγ ELISA was performed. IFNγ levels at different co-culture conditions are indicated on the graph.

FIG. 8 Effector function of the identified Cb1 precision paired mutation can be transferred to TCRs with other specificities. IFN-γ ELISA of supernatants derived from the co-cultures of NY-ESO-mut_4.3Cb1 TCR T cells for 20 hours with target cells (Mel624.38 tumor cells expressing NY-ESO) at an E:T ratio of 2:1 were performed. As a control, wildtype NY-ESO TCR expressing T cells (NY-ESO wt) and minimally murinized NY-ESO TCR expressing T cells were also tested. Positive control comprised effector cells activated with 750 ng/ml PMA and 5 ng/ml ionomycin (PMA/lono). Untransduced cells (T cells only) served as negative control.

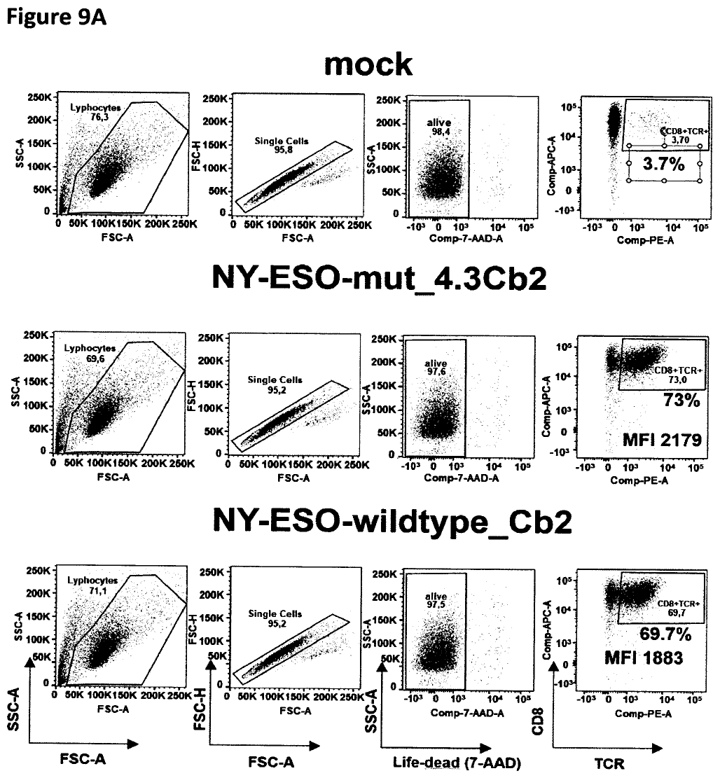

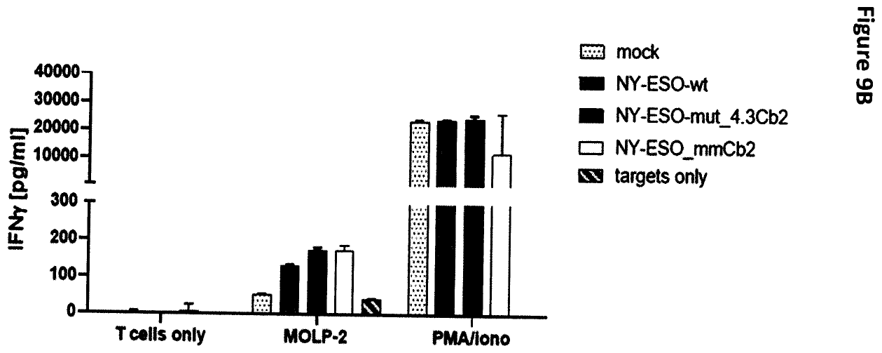

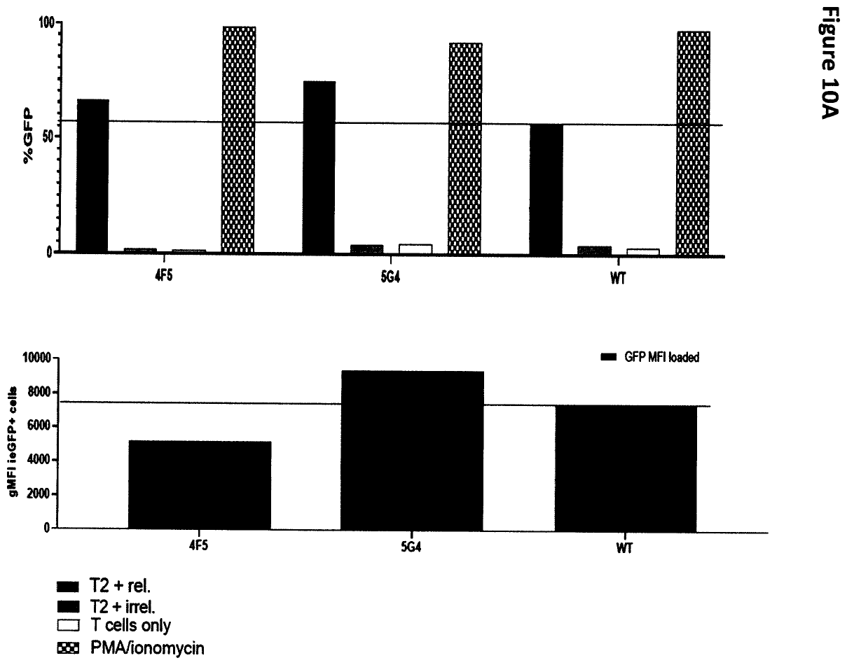

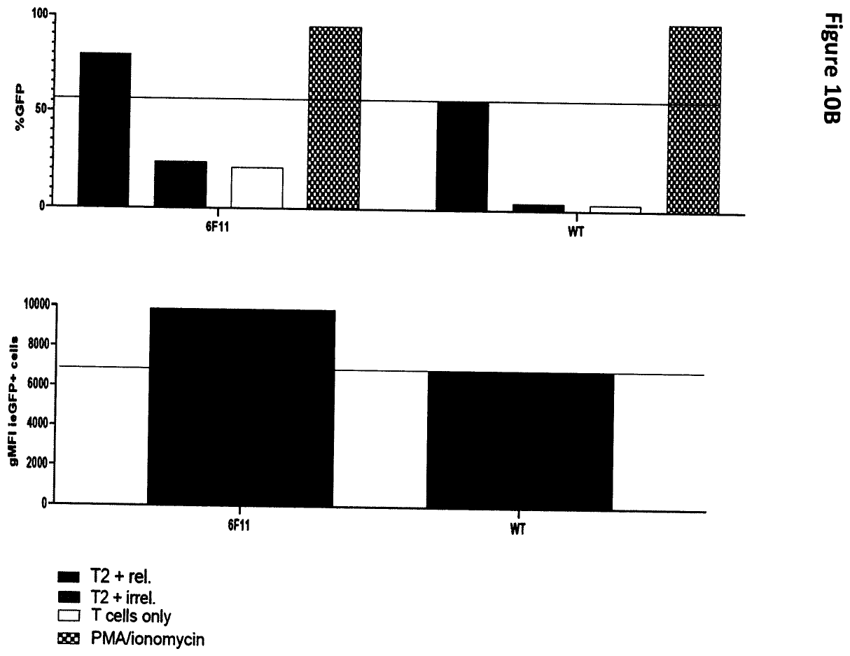

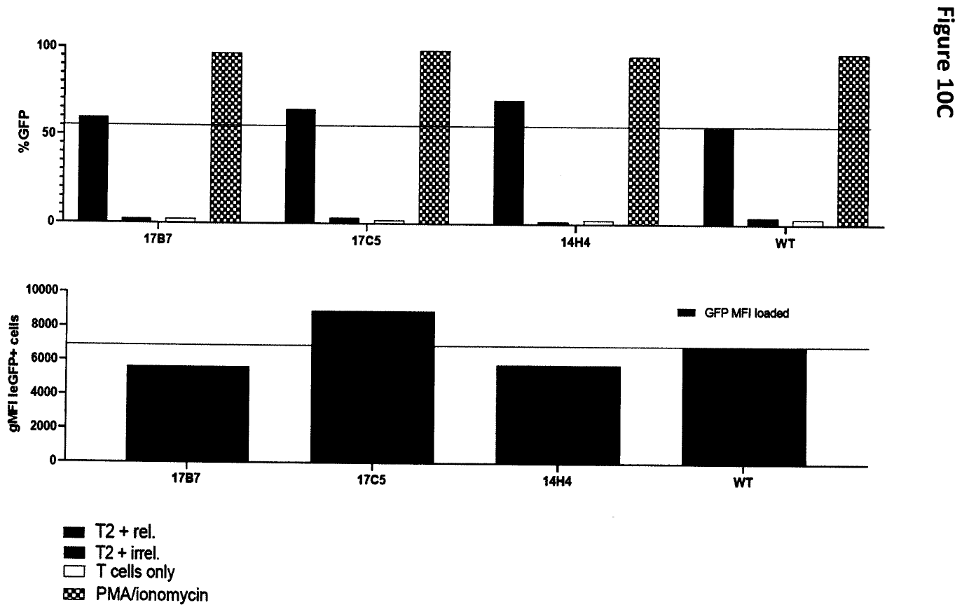

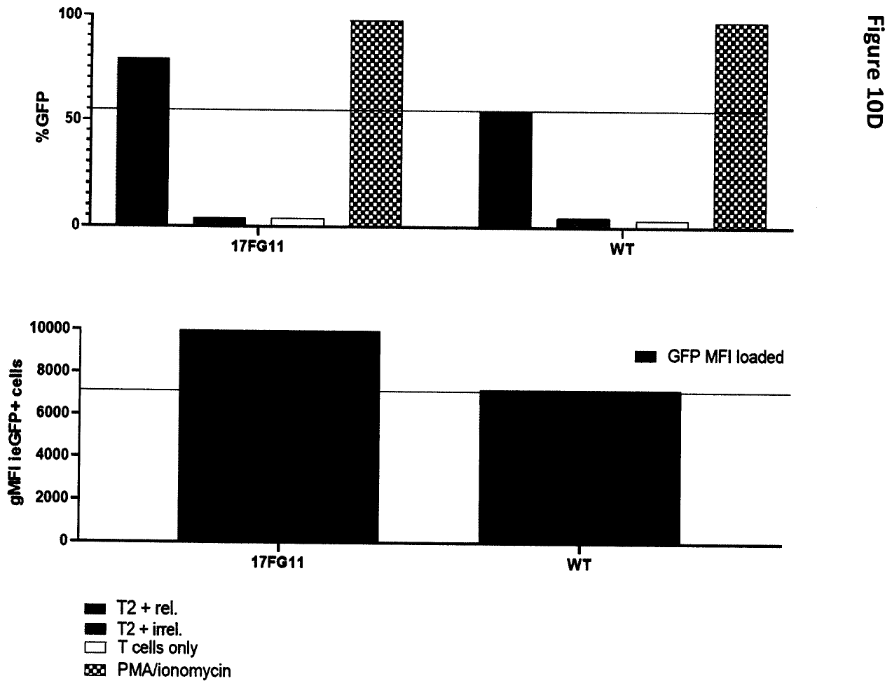

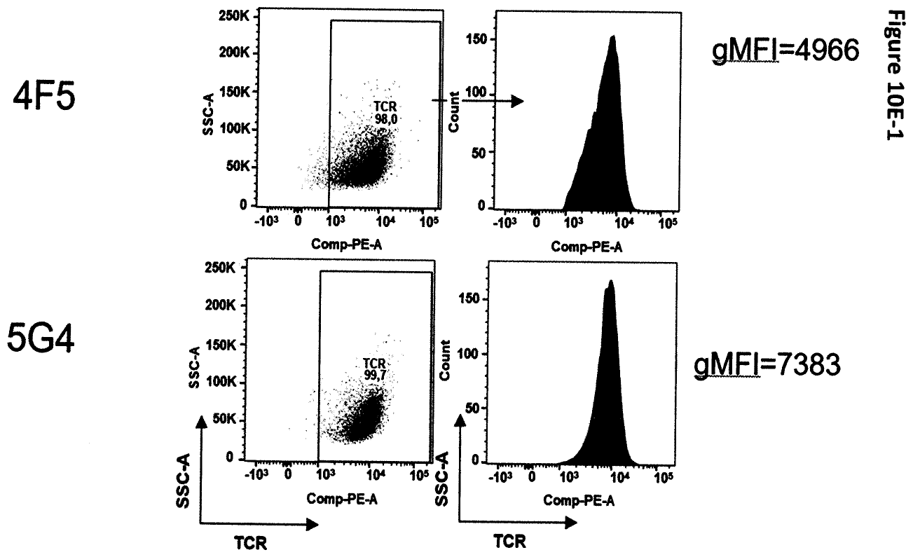

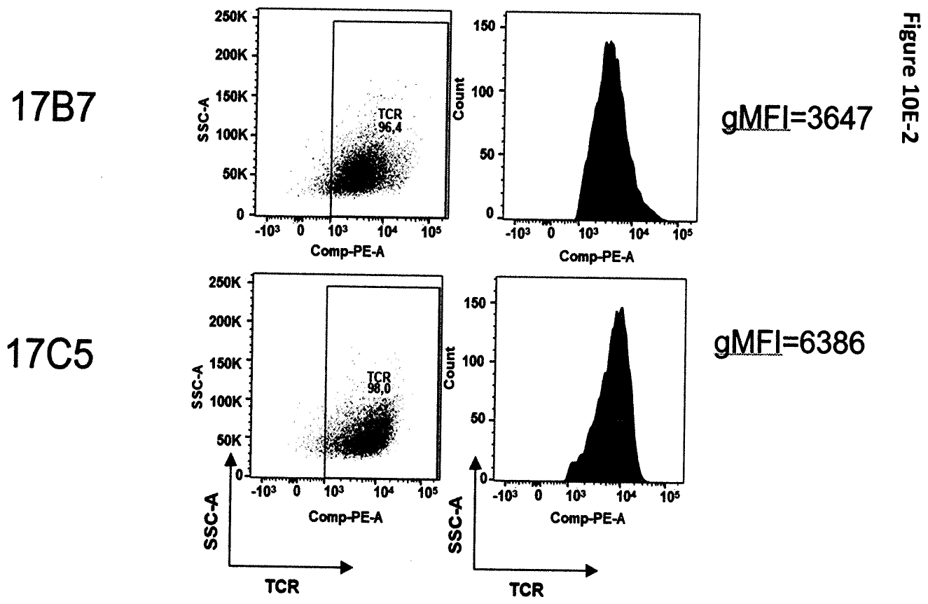

FIG. 9 Effector function of the identified Cb1 precision paired mutation can be transferred to Cb2 allele of the same TCR. (A) Staining of wildtype TCR T cells and PP mutated NY-ESO-TCR CD8+ T cells using anti-CD8 antibody [APC, clone SK1, BD Bioscience] and anti TCR Vβ1 (TRBV9) antibody [PE, clone BL37.2, Beckman Coulter] followed by flow cytometric analysis to compare the TCR surface expression. Untransduced T cells were used as respective negative controls. (B) IFN-γ ELISA of supernatants derived from the 20 hours co-cultures (E: T ratio 2:1) of NY-ESO-mut_4.3Cb2 TCR T cells with MOLP-2 tumor cells expressing NY-ESO. As a control, wildtype NY-ESO TCR expressing T cells (NY-ESO wt) and minimally murinized NY-ESO TCR (NY-ESO_mmCb2) expressing T cells were also tested. Positive control comprised effector cells activated with 750 ng/ml PMA and 5 ng/ml ionomycin (PMA/lono). Mock transduced cells served as negative control.

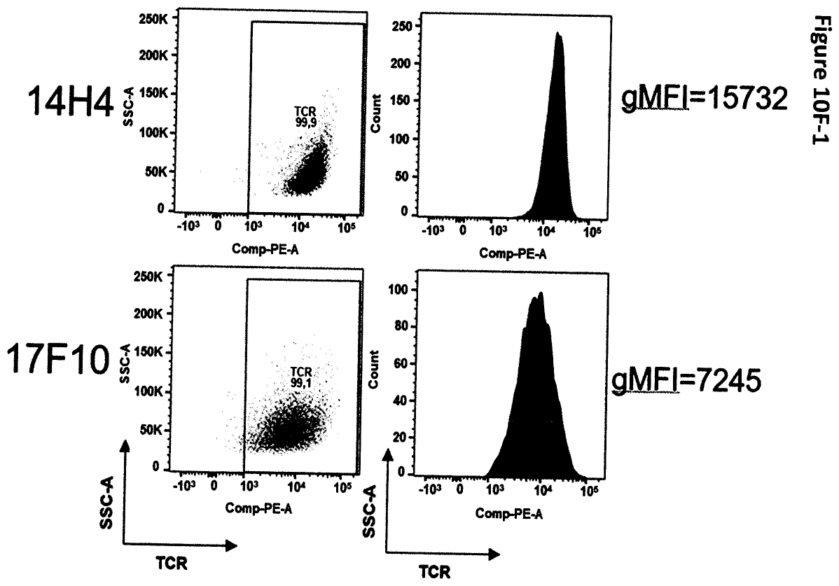

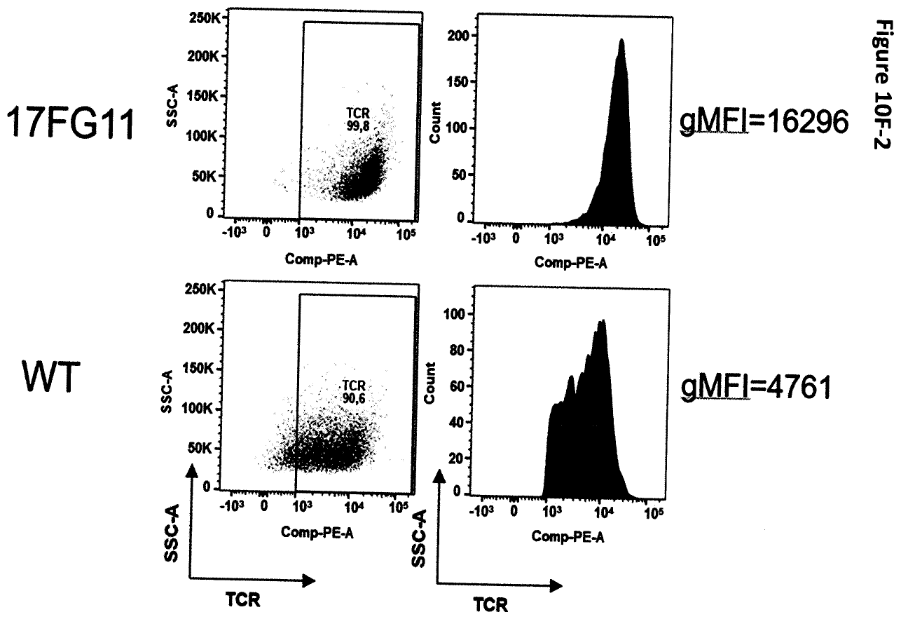

FIG. 10 Testing TCR specificity of selected clones. (A-D) Several clones (clone names given below x-axis) and WT were co-cultured with T2 cells loaded with either relevant VLDGLDVLL peptide (T2+rel.), irrelevant peptide SLLQHLIGL (T2+irrel.) or left alone (T cells only). After 24 h of co-culture, cells were collected and percentage of eGFP positive cells and gMFI of eGFP signal on the transduced effector cells was measured with flow cytometry, gMFI of eGFP channel was plotted. (E-F) Expression of precision paired TCRs in TCR-deficient Jurkat-76 CD+ieGFP cells. Transduced Jurkat-76 cells were analyzed for TCR expression by staining with anti TCR Vβ1 (TRBV9) antibody [PE, clone BL37.2, Beckman Coulter] antibody and subsequent flow cytometric analysis.

### DETAILED DESCRIPTION OF THE INVENTION

Before the invention is described in detail with respect to some of its preferred embodiments, the following general definitions are provided.

The present invention as illustratively described in the following may suitably be practiced in the absence of any element or elements, limitation or limitations, not specifically disclosed herein.

The present invention will be described with respect to particular embodiments and with reference to certain figures, but the invention is not limited thereto but only by the claims.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group which preferably consists only of these embodiments.

Where an indefinite or definite article is used when referring to a singular noun, e.g. “a”, “an” or “the”, this includes a plural of that noun unless something else is specifically stated.

Technical terms are used by their common sense. If a specific meaning is conveyed to certain terms, definitions of terms will be given in the following in the context of which the terms are used.

A first aspect of the invention relates to a library of synthetic polynucleotides
encoding TCR alpha chain constant regions and TCR beta chain constant regions, wherein the library comprises:
   polynucleotides encoding TCR alpha chain constant regions which are selected from the group of TCR alpha constant regions which are at least about 80% identical to the amino acid sequence as set out in SEQ ID NO: 1;
   polynucleotides encoding TCR beta chain constant regions which are selected from the group of TCR beta constant regions which are at least about 80% identical to the amino acid sequence as set out in SEQ ID NO: 2.

SEQ ID NO: 1 defines TCR alpha chain constant regions which are set out in following amino acid sequence:

IQNPDPAVYQLRDXKSSDKSVCLFTDFDSQTNVSQSKDSDVYITDKTVLD
MRSMDFKSNSAVAWSXKSDFACANAFNNSIIPEDTFFPSPEXXCDVKLVE
KSFETDXNLNFQNLXVIXXRILLLKVAGFNLLMTLRLWSS

wherein
X at position 14 is selected from the group consisting of S, V, W, A, C, F, G, I, L, M and P;
X at position 66 is selected from the group consisting of N, Q, S, T and Y;
X at position 92 is selected from the group consisting of S, V, W, A C, F, G, I, L, M and P;
X at position 93 is selected from the group consisting of S, V, W, A C, F, G, I, L, M and P;
X at position 107 is selected from the group consisting of T, V, W, A, C, F, G, I, L, M and P;
X at position115 is selected from the group consisting of S, V, W, A, C, F, G, I, L, M and P;
X at position 118 is selected from the group consisting of G, I, L, M, P, V, W, A, C and F; and
X at position 119 is selected from the group consisting of F, G, I, L, M, P, V, W, A and C;
The skilled person understands that in SEQ ID NO: 1 X at position 14 corresponds to X01, X at position 66 corresponds to X02, X at position 92 corresponds to X03, X at position 93 corresponds to X04, X at position 107 corresponds to X05, X at position 115 corresponds to X06, X at position 118 corresponds to X07, X at position 119 corresponds to position X08 in FIG. 1A.

SEQ ID NO: 2 defines TCR beta chain constant regions (including both Cbeta1 and Cbeta2 versions) which are set out in following amino acid sequence:

EDLXXVFPPEVAVFEPSXAEIXHTQKATLVCLAXGFXPDHVELSWWVNGK
EVHSGVSTDPQPLKEQPALNDSRYCLSSRLRVSATFWQNPRNHFRCQVQF
YGLSENDEWTQDRAKPVTQIVSAEAWGRADCGFTSXSYQQGVLSATILYE
ILLGKAXLYAVLVXALVLMAMVKRKDXXX

wherein
X at position 4 is selected from the group consisting of K, N;
X at position 5 is selected from the group consisting of N, R, D, E, H and K;
X at position 18 is selected from the group consisting of E, H, K, R and D;
X at position 22 is selected from the group consisting of S, V, W, A, C, F, G, I, L, M and P;
X at position 34 is selected from the group consisting of T, D, E, H, K, N and R;
X at position 37 is selected from the group consisting of Y, F;
X at position 136 is selected from the group consisting of E, V;
X at position 157 is selected from the group consisting of T, V, W, A, C, F, G, I, L, M and P; and
X at position 164 is selected from the group consisting of S, V, W, A, C, F, G, I, L, M and P;
X at position 177 is selected from the group consisting of S, F;
X at position 178 is R or deleted;
X at position 179 is G or deleted.
The skilled person understands that in SEQ ID NO 2, X at position 5 corresponds to X09, X at position 18 corresponds to X10, X at position 22 corresponds to X11, X at position 34 corresponds to X12, X at position 157 corresponds to X13, X at position 164 corresponds to X14 in FIG. 1B.
X at position 4, 37, 136, 177, 178, 179 covers both variations for Cbeta 1 and Cbeta

Where a sequence is defined as being to a particular percentage identical to an amino acid sequence as set out herein, the amino acids at the specific variable positions identified by the invention (e.g. denotated as X01 etc.) can only be selected from the amino acids as defined herein. In other words, at the variable position according to the invention, only the indicated alternative amino acid sequences can be selected.

Applying to all aspects of the invention, in some embodiments, the TCR alpha chain constant regions are selected from the group of TCR alpha constant regions which are at least about 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and the TCR beta chain constant regions are selected from the group of TCR beta constant regions which are at least about 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO.

More specifically, in some embodiments the polynucleotides encoding TCR alpha chain constant regions are selected from the group of TCR alpha constant regions which are at least about 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1.

In some embodiments the polynucleotides encoding TCR beta chain constant regions are selected from the group of TCR beta constant regions which are at least about 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 2.

In some embodiments the polynucleotides encoding TCR beta chain constant regions are selected from the group of TCR beta constant regions which are at least about 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 15.

The constant region of the TCR beta chain occurs naturally either in the Cbeta1 version or in the Cbeta2 version, which are typically equally distributed in a population of natural unstimulated T cells. The skilled person understands, that the invention refers to both versions of the constant region. Thus, the TCR beta chain constant regions may be a Cbeta1 version as set out in SEQ ID NO: 15 or a Cbeta 2 version as set out in SEQ ID NO: 16. Thus, TCR beta chain constant regions comprising constant regions which are selected from the group of TCR beta constant regions which are at least about 80% identical to the amino acid sequence as set out in SEQ ID NO 2 cover both Cbeta1 and Cbeta2 versions.

SEQ ID NO: 15 defines TCR beta chain constant regions (Cbeta1) which are set out in following amino acid sequence:

EDLNXVFPPEVAVFEPSXAEIXHTQKATLVCLAXGFFPDHVELSWWVNG
KEVHSGVSTDPQPLKEQPALNDSRYCLSSRLRVSATFWQNPRNHFRCQ
VQFYGLSENDEWTQDRAKPVTQIVSAEAWGRADCGFTSVSYQQGVLSA
TILYEILLGKAXLYAVLVXALVLMAMVKRKDF

wherein
X at position 5 is selected from the group consisting of N, R, D, E, H and K;
X at position 18 is selected from the group consisting of E, H, K, R and D;
X at position 22 is selected from the group consisting of S, V, W, A, C, F, G, I, L, M and P;
X at position 34 is selected from the group consisting of T, D, E, H, K, N and R;
X at position 157 is selected from the group consisting of T, V, W, A, C, F, G, I, L, M and P; and
X at position 164 is selected from the group consisting of S, V, W, A, C, F, G, I, L, M and P;

The skilled person understands that for SEQ ID NO: 15 X09 to X14 are the identical positions as set out for SEQ ID NO: 2 in FIG. 1B. Thus, In SEQ ID NO: 15, X at position 5 corresponds to X09, X at position 18 corresponds to X10, X at position 22 corresponds to X11, X at position 34 corresponds to X12, X at position 157 corresponds to X13, X at position 164 corresponds to X14 in FIG. 1B.

SEQ ID NO: 16 defines TCR beta chain constant regions (Cbeta2) which are set out in following amino acid sequence:

EDLKXVFPPEVAVFEPSXAEIXHTQKATLVCLAXGFYPDHVELSWWVNG
KEVHSGVSTDPQPLKEQPALNDSRYCLSSRLRVSATFWQNPRNHFRCQ
VQFYGLSENDEWTQDRAKPVTQIVSAEAWGRADCGFTSESYQQGVLSA
TILYEILLGKAXLYAVLVXALVLMAMVKRKDSRG

wherein
X at position 5 is selected from the group consisting of N, R, D, E, H and K;
X at position 18 is selected from the group consisting of E, H, K, R and D;
X at position 22 is selected from the group consisting of S, V, W, A, C, F, G, I, L, M and P;
X at position 34 is selected from the group consisting of T, D, E, H, K, N and R;
X at position 157 is selected from the group consisting of T, V, W, A, C, F, G, I, L, M and P; and
X at position 164 is selected from the group consisting of S, V, W, A, C, F, G, I, L, M and P;

In SEQ ID NO: 16, X at position 5 corresponds to X09, X at position 18 corresponds to X10, X at position 22 corresponds to X11, X at position 34 corresponds to X12, X at position 157 corresponds to X13, X at position 164 corresponds to X14 in FIG. 1B.

In one embodiment refers to a library of synthetic polynucleotides encoding TCR alpha chain constant regions and TCR beta chain constant regions, wherein the library comprises:
polynucleotides encoding TCR alpha chain constant regions which are selected from the group of TCR alpha constant regions comprising the amino acid sequence as set out in SEQ ID NO: 1;
polynucleotides encoding TCR beta chain constant regions which are selected from the group of TCR beta constant regions comprising the amino acid sequence as set out in SEQ ID NO: 15; or polynucleotides encoding TCR beta chain constant regions which are selected from the group of TCR beta constant regions comprising the amino acid sequence as set out in SEQ ID NO: 16.

The determination of percent identity between multiple sequences is preferably accomplished using the AlignX application of the Vector NTI Advance™ 10 program (Invitrogen Corporation, Carlsbad CA, USA). This program uses a modified Clustal W algorithm (Thompson et al., 1994. Nucl Acids Res. 22: pp. 4673-4680; Invitrogen Corporation; Vector NTI Advance™ 10 DNA and protein sequence analysis software. User's Manual, 2004, pp. 389-662). The determination of percent identity is performed with the standard parameters of the AlignX application.

Accordingly, the invention also relates to a library of synthetic polynucleotides encoding TCR alpha chains and TCR beta chains, comprising
Polynucleotides encoding TCR alpha chains comprising constant regions which are selected from the group of TCR alpha constant regions which are at least about 80% identical to the amino acid sequence as set out in SEQ ID NO: 1;
Polynucleotides encoding TCR beta chains comprising constant regions which are selected from the group of TCR beta constant regions which are at least about 80% identical to the amino acid sequence as set out in SEQ ID NO: 15; or
Polynucleotides encoding TCR beta chains comprising constant regions which are selected from the group of TCR beta constant regions which are at least about 80% identical to the amino acid sequence as set out in SEQ ID NO: 16.

In some embodiments the polynucleotides encoding TCR alpha chains comprising constant regions are selected from the group of TCR alpha constant regions which are at least about 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1.

In some embodiments the polynucleotides encoding TCR beta chains comprising constant regions are selected from the group of TCR beta constant regions which are at least about 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 2.

In some embodiments the polynucleotides encoding TCR beta chains comprising constant regions are selected from the group of TCR beta constant regions which are at least about 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 15.

A specific embodiment refers to a library of synthetic polynucleotides encoding TCR alpha chains and TCR beta chains, comprising
Polynucleotides encoding TCR alpha chains comprising constant regions which are selected from the group of TCR alpha constant regions which comprise the amino acid sequence of SEQ ID NO: 1;
Polynucleotides encoding TCR beta chains comprising constant regions which are selected from the group of TCR beta constant regions which comprise the amino acid sequence of SEQ ID NO: 15, or
Polynucleotides encoding TCR beta chains comprising constant regions which are selected from the group of TCR beta constant regions which comprise the amino acid sequence of SEQ ID NO: 16.

“Nucleic acid” generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. Preferably, the nucleic acids described herein are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication. The nucleic acids can be constructed based on chemical synthesis and/or enzymatic ligation reactions using procedures known in the art or commercially available (e.g. from GeneArt, Thermo Fisher and similar companies). See, for example Sambrook et al., a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). The nucleic acid can comprise any nucleotide sequence which encodes any of the recombinant TCRs, polypeptides, or proteins, or functional portions or functional variants thereof.

The selection marker may be any useful selection marker, such as antibiotic resistance, fluourescent marker, binding epitope. The selection marker may be thus selected from, but not limited to, CD20 or Her2/neu markers, or other conventional tags such as a myc-tag, FLAG-tag, T7-tag, HA (hemagglutinin)-tag, His-tag, S-tag, GST-tag, myc, T7, GST, or fluorescent marker, such as GFP, BFP, mTagBFP.

A TCR is composed of two different and separate protein chains, namely the TCR alpha (a) and the TCR beta (B) chain. The TCR α chain comprises variable region (encoded by the variable (V), joining (J) genomic segments) and constant region (encoded by the constant genomic segment). The TCR β chain comprises the variable region (encoded by the variable (V), diversity (D), joining (J) genomic segments) and constant region (encoded by the constant (C) genomic segment). The rearranged V (D) J regions of both the TCR α and the TCR β chain contain hypervariable regions (CDR, complementarity determining regions), among which the CDR3 region determines the specific epitope recognition. At the C-terminal region both TCR α chain and TCR β chain contain a hydrophobic transmembrane domain and end in a short cytoplasmic tail.

Typically, the library contains TCR alpha chains, which contain only variations in their constant region within one library. The remaining part, i.e. the variable region of the TCR is identical within one library. Accordingly, the library contains TCR beta chains, which contain only variations in their constant region within one library. The remaining part, i.e. the variable region of the TCR is identical within one library.

In one embodiment the library comprises polynucleotides encoding a TCR alpha chain and a TCR beta chain, each polynucleotide comprising:
Polynucleotide encoding TCR alpha chains as defined herein and
Polynucleotide encoding TCR beta chains as defined herein;
Optionally a selection marker
Optionally one or more ribosomal entry sites or self-cleaving peptides of the 2A family.

The polynucleotide may contain one or more internal ribosomal entry sites (IRES) sequence or self-cleaving peptides of the 2A family, comprising the 2A peptide sequence derived from a porcine tsechovirus (P2A), derived from other species like Thosea asigna virus 2A peptide (T2A), derived from foot and mouth disease virus 2A peptide (F2A) (as described in Szymczak et al.: Development of 2A peptide-based strategies in the design of multicistronic vectors) and E2A, resulting in the expression a single messenger RNA (mRNA) molecule under the control of the viral promoter within the transduced cell.

The term “library” refers to a collection of distinct molecules comprising typically more than 10³, more than 10⁴, more than 10⁵, more than 10⁶, more than 10⁷, more than 10⁸, more than 10⁹ or even more than 10″ members. A library in the context of the present invention is a mixture of heterogeneous polypeptides or nucleic acids. The library is composed of members, each of which has a single polypeptide or nucleic acid sequence. Sequence differences between library members are responsible for the diversity present in the library. The library may take the form of a simple mixture of polypeptides or nucleic acids, or may be in the form of cells containing the library of nucleic acids. Preferably, a cell contains only one or a limited number of library members. Advantageously, the nucleic acids are incorporated into expression vectors, in order to allow expression of the polypeptides encoded by the nucleic acids.

Typically, the library comprises at least 1×10⁴, at least 1×10⁵, at least 1×10⁶, at least 1×10⁷, at least 1×10⁸, at least 1×10⁹, preferably, 1×10¹⁰, more preferably 2.1×10¹¹, such as 2.25×10¹³ unique molecules. Preferably, the library has 1×10⁶ to 1×10¹⁵, more preferably 1×10¹⁰ to 1×10¹⁴ unique molecules. Comprising a number of unique molecules means that each of these molecules of this number differs to each of the other molecules of this number. In one embodiment “unique molecules” means that these molecules exist only once in the library. It should be understood that there is likely to be more than one copy of many unique molecules in a particular physical realization.

The data shows that different constant regions selected from the library show improved expression on the cell surface:

| SEQ ID NO: | Description |
|---|---|
| 12 | Constant region 8-9 alpha chain |
| 13 | Constant region 8-9 beta chain |
| 17 | Constant region 1 (4F5) alpha chain |
| 18 | Constant region 1 (4F5) beta chain |
| 19 | Constant region 2 (5G4) alpha chain |
| 20 | Constant region 2 (5G4) beta chain |
| 21 | Constant region 3 (6G9) alpha chain |
| 22 | Constant region 3 (6G9) beta chain |
| 23 | Constant region 4 (17C5) alpha chain |
| 24 | Constant region 4 (17C5) beta chain |
| 25 | Constant region 13 (17FG11) alpha chain |
| 26 | Constant region 13 (17FG11 beta chain |
| 27 | Constant region 14 (17B7) alpha chain |
| 28 | Constant region 14 (17B7) beta chain |
| 29 | Constant region 15 (14H4) alpha chain |
| 30 | Constant region 15 (14H4) beta chain |
| 31 | Constant region 16 (14H3) alpha chain |
| 32 | Constant region 16 (14H3)beta chain |

Hence the constant regions as set out in SEQ ID NOs: 12, 13, and 17 to 32 and any combination of alpha and beta constant regions thereof are also embodiments of the invention.

Thus, in some embodiments, a TCR alpha chain constant region may comprise an amino acid sequence which is at least about 80% identical to the amino acid sequence selected from the group consisting of SEQ ID NO: 12, 17, 19, 21, 23, 25, 27, 29 and 31 wherein the amino acids at position 14, 66, 92, 93, 107, 115, 118 and 119 are not changed. Accordingly, a TCR beta chain constant region may comprise an amino acid sequence which is at least about 80% identical to the amino acid sequence selected from the group consisting of SEQ ID NO: 13, 18, 20, 22, 24, 26, 28, 30 and 32 wherein the amino acids at position 5, 18, 22, 34, 157 and 164 are not changed.

The TCR alpha chain constant regions and the TCR beta chain constant region are selected from the following:
a) TCR alpha chain constant region comprising an amino acid sequence which is at least about 80% identical to the amino acid sequence as set out in SEQ ID NO: 12 wherein the amino acids at position 14, 66, 92, 93, 107, 115, 118 and 119 are not changed and a TCR beta chain constant region comprising an amino acid sequence which is at least about 80% identical to the amino acid sequence as set out in SEQ ID NO: 13, wherein the amino acids at position 5, 18, 22, 34, 157 and 164 are not changed;
b) TCR alpha chain constant region comprising an amino acid sequence which is at least about 80% identical to the amino acid sequence as set out in SEQ ID NO: 17 wherein the amino acids at position 14, 66, 92, 93, 107, 115, 118 and 119 are not changed and a TCR beta chain constant region comprising an amino acid sequence which is at least about 80% identical to the amino acid sequence as set out in SEQ ID NO: 18, wherein the amino acids at position 5, 18, 22, 34, 157 and 164 are not changed;
c) TCR alpha chain constant region comprising an amino acid sequence which is at least about 80% identical to the amino acid sequence as set out in SEQ ID NO: 19 wherein the amino acids at position 14, 66, 92, 93, 107, 115, 118 and 119 are not changed and a TCR beta chain constant region comprising an amino acid sequence which is at least about 80% identical to the amino acid sequence as set out in SEQ ID NO: 20, wherein the amino acids at position 5, 18, 22, 34, 157 and 164 are not changed;
d) TCR alpha chain constant region comprising an amino acid sequence which is at least about 80% identical to the amino acid sequence as set out in SEQ ID NO: 21 wherein the amino acids at position 14, 66, 92, 93, 107, 115, 118 and 119 are not changed and a TCR beta chain constant region comprising an amino acid sequence which is at least about 80% identical to the amino acid sequence as set out in SEQ ID NO: 22, wherein the amino acids at position 5, 18, 22, 34, 157 and 164 are not changed;
e) TCR alpha chain constant region comprising an amino acid sequence which is at least about 80% identical to the amino acid sequence as set out in SEQ ID NO: 23 wherein the amino acids at position 14, 66, 92, 93, 107, 115, 118 and 119 are not changed and a TCR beta chain constant region comprising an amino acid sequence which is at least about 80% identical to the amino acid sequence as set out in SEQ ID NO: 24, wherein the amino acids at position 5, 18, 22, 34, 157 and 164 are not changed;
f) TCR alpha chain constant region comprising an amino acid sequence which is at least about 80% identical to the amino acid sequence as set out in SEQ ID NO: 25 wherein the amino acids at position 14, 66, 92, 93, 107, 115, 118 and 119 are not changed and a TCR beta chain constant region comprising an amino acid sequence which is at least about 80% identical to the amino acid sequence as set out in SEQ ID NO: 26, wherein the amino acids at position 5, 18, 22, 34, 157 and 164 are not changed;
g) TCR alpha chain constant region comprising an amino acid sequence which is at least about 80% identical to the amino acid sequence as set out in SEQ ID NO: 27 wherein the amino acids at position 14, 66, 92, 93, 107, 115, 118 and 119 are not changed and a TCR beta chain constant region comprising an amino acid sequence which is at least about 80% identical to the amino acid sequence as set out in SEQ ID NO: 28, wherein the amino acids at position 5, 18, 22, 34, 157 and 164 are not changed;
h) TCR alpha chain constant region comprising an amino acid sequence which is at least about 80% identical to the amino acid sequence as set out in SEQ ID NO: 29 wherein the amino acids at position 14, 66, 92, 93, 107, 115, 118 and 119 are not changed and a TCR beta chain constant region comprising an amino acid sequence which is at least about 80% identical to the amino acid sequence as set out in SEQ ID NO: 30, wherein the amino acids at position 5, 18, 22, 34, 157 and 164 are not changed;
i) TCR alpha chain constant region comprising an amino acid sequence which is at least about 80% identical to the amino acid sequence as set out in SEQ ID NO: 32 wherein the amino acids at position 14, 66, 92, 93, 107, 115, 118 and 119 are not changed and a TCR beta chain constant region comprising an amino acid sequence which is at least about 80% identical to the amino acid sequence as set out in SEQ ID NO: 32, wherein the amino acids at position 5, 18, 22, 34, 157 and 164 are not changed;

In specific embodiments the TCR alpha chain constant regions and the TCR beta chain constant region are selected from the following:
a) TCR alpha chain constant region comprising the amino acid sequence as set out in SEQ ID NO: 12 and a TCR beta chain constant region comprising the amino acid sequence as set out in SEQ ID NO: 13;
b) TCR alpha chain constant region comprising the amino acid sequence as set out in SEQ ID NO: 17 and a TCR beta chain constant region comprising the amino acid sequence as set out in SEQ ID NO: 18;
c) TCR alpha chain constant region comprising the amino acid sequence as set out in SEQ ID NO: 19 and a TCR beta chain constant region comprising the amino acid sequence as set out in SEQ ID NO: 20;
d) TCR alpha chain constant region comprising the amino acid sequence as set out in SEQ ID NO: 21 and a TCR beta chain constant region comprising the amino acid sequence as set out in SEQ ID NO: 22;
e) TCR alpha chain constant region comprising the amino acid sequence as set out in SEQ ID NO: 23 and a TCR beta chain constant region comprising the amino acid sequence as set out in SEQ ID NO: 24;
f) TCR alpha chain constant region comprising the amino acid sequence as set out in SEQ ID NO: 25 and a TCR beta chain constant region comprising the amino acid sequence as set out in SEQ ID NO: 26;
g) TCR alpha chain constant region comprising the amino acid sequence as set out in SEQ ID NO: 27 and a TCR beta chain constant region comprising the amino acid sequence as set out in SEQ ID NO: 28;
h) TCR alpha chain constant region comprising the amino acid sequence as set out in SEQ ID NO: 29 and a TCR beta chain constant region comprising the amino acid sequence as set out in SEQ ID NO: 30;
i) TCR alpha chain constant region comprising the amino acid sequence as set out in SEQ ID NO: 31 and a TCR beta chain constant region comprising the amino acid sequence as set out in SEQ ID NO: 32;

Also encompassed is a library of vectors comprising the polynucleotide library as defined herein.

A “vector” is any molecule or composition that has the ability to carry a nucleic acid sequence into a suitable host cell where synthesis of the encoded polypeptide can take place. Typically, and preferably, a vector is a nucleic acid that has been engineered, using recombinant DNA techniques that are known in the art, to incorporate a desired nucleic acid sequence (e.g. a nucleic acid of the invention). The vector may comprise DNA or RNA and/or comprise liposomes. The vector may be a plasmid, shuttle vector, phagemide, cosmid, expression vector, retroviral vector, lentiviral vector, adenoviral vector or particle. A vector may include nucleic acid sequences that permit it to replicate in a host cell, such as an origin of replication. A vector may also include one or more selectable marker genes and other genetic elements known to those of ordinary skill in the art. A vector preferably is an expression vector that includes a nucleic acid according to the present invention operably linked to sequences allowing for the expression of said nucleic acid.

Preferably, the vector is a retroviral particle.

Further encompassed is a library of cells, comprising said library of vectors. Moreover, also a library of cells expressing the polynucleotides as defined herein is encompassed.

Thus, in the library of cells the above described vector comprising a nucleic acid sequence coding for the above described TCR may be introduced or the nucleic acid may be introduced by other means, e.g. In vitro transcribed RNA (ivtRNA) coding for said TCR may be introduced.

The cell may be a peripheral blood lymphocyte such as a T cell. The method of cloning and exogenous expression of the TCR is for example described in Engels et al. (Relapse or eradication of cancer is predicted by peptide-major histocompatibility complex affinity. Cancer Cell, 23 (4), 516-26. 2013). The transduction of primary human T cells with a lentiviral vector is, for example, described in Cribbs “simplified production and concentration of lentiviral vectors to achieve high transduction in primary human T cells” BMC Biotechnol. 2013; 13:98.

The term “transduction” refers to the process by which an exogenous nucleic acid sequence is introduced into a host cell, e.g. into a T cell. It is noted that introduction or transfer of nucleic acid sequences is not limited to the mentioned methods but can be achieved by any number of means including electroporation, microinjection, gene gun delivery, lipofection, superfection, infection by retroviruses or other suitable viruses for transduction or transfection.

Preferably the cell may be a T cell. The T cell may express endogenous TCR chains. The T cell may be a CD4+ or a CD8+ T cell. Preferably, the T cell is a CD8+ T cell. In one embodiment the cell may be a Jurkat-ieGFP TCR−/−CD8+ cell.

A further aspect relates to a library of polypeptides encoded by the library of synthetic polynucleotides as defined herein.

Another aspect refers to the use of the library as described herein for identifying a TCR receptor with enhanced TCR alpha and TCR beta chain pairing.

A further aspect refers to a method for isolating a TCR with enhanced TCR alpha and beta chain pairing, comprising the steps:
Co-culturing the library of cells as described herein with antigen presenting cells presenting the HLA bound form of the target antigen of the TCR;
and at least one of the steps:
Selecting cells showing elevated expression levels of the heterologous TCR compared to wildtype, and/or
Selecting cells showing elevated activation levels compared to wild type.

Wildtype with regard to “compared to wildtype” means in particular a TCR which is identical in the amino acid sequence to the TCR it is compared except that it contains in all positions X01 to X14 the wildtype amino acid. The skilled person is aware that the wild type amino acids for X01 to X014 regarding the Cbeta2 chain are as follows:
X01═S; X02=N; X03═S; X04═S; X05=T; X06═S; X07=G; X08=F; X09=N;
X10=E; X11═S; X12=T; X13=T and X14=S. The skilled person is aware that the wild type amino acids for X01 to X014 regarding the Cbeta1 chain are as follows:
X01═S; X02=N; X03═S; X04═S; X05=T; X06═S; X07=G; X08=F; X09=K;
X10=E; X11═S; X12=T; X13=T and X14=S.

Accordingly also a TCR isolated from the TCR library as described herein is encompassed.

Preferably, the TCR isolated from the TCR library shows elevated expression levels of the heterologous TCR compared to wild type and/or elevated activation levels compared to wild type.

Thus, another aspect of the invention refers to a TCR (and nucleic acids and vectors encoding such TCR), comprising
A TCR alpha chain comprising constant region which is at least about 80% identical to the amino acid sequence as set out in SEQ ID NO: 1;
TCR beta chains comprising a constant regions which is at least about 80% identical to the amino acid sequence as set out in SEQ ID NO: 15; or
TCR beta chains comprising a constant regions which is at least about 80% identical to the amino acid sequence as set out in SEQ ID NO: 16.

In one embodiment, at least one of the variable positions, i.e. X01 to X14, the amino acid is not the amino acid which occurs naturally in wild type. That means that preferably in at least two, more preferably at least three, even more preferably at least four, such as at least five, at least 6, at least 7, at least 8, at least 9, at least 10, at least 11, at least 12, at least 13, such as in all 14 positions of X01 to X14, the amino acid is not the amino acid which occurs naturally in wild type. The term “not the amino acid which occurs naturally in wild type” means in the context of this application that it is substituted by an amino acid as defined for the variable positions X1 to X14, which is not the wild type amino acid for this position. The skilled person is aware that the wild type amino acids for X01 to X014 regarding the Cbeta2 chain are as follows: X01═S; X02=N; X03═S; X04═S; X05=T; X06═S; X07=G; X08=F; X09=N; X10=E; X11═S; X12=T; X13=T and X14=S. The skilled person is aware that the wild type amino acids for X01 to X014 regarding the Cbeta1 chain are as follows: X01═S; X02=N; X03═S; X04═S; X05=T; X06═S; X07=G; X08=F; X09=K; X10=E; X11═S; X12=T; X13=T and X14=S.

The TCRs according to the invention may show elevated expression levels of the heterologous TCR compared to wild type. Alternatively, or in addition, the TCRs according to the invention may show elevated activation levels compared to wild type. Preferably the TCRs according to the invention show elevated expression levels of the heterologous TCR compared to wild type and show elevated activation levels compared to wild type.

Another aspect refers to a library of TCR alpha chains and TCR beta chains, comprising
TCR alpha chains comprising constant regions which are selected from the group of TCR alpha constant regions which are at least about 80% identical to the amino acid sequence as set out in SEQ ID NO: 1;
TCR beta chains comprising constant regions which are selected from the group of TCR beta constant regions which are at least about 80% identical to the amino acid sequence as set out in SEQ ID NO: 15;
TCR beta chains comprising constant regions which are selected from the group of TCR beta constant regions which are at least about 80% identical to the amino acid sequence as set out in SEQ ID NO: 16.

Thus, one embodiment refers to a library of TCR alpha chains and TCR beta chains, comprising
TCR alpha chains comprising constant regions which are selected from the group of TCR alpha constant regions which comprise the amino acid sequence of SEQ ID NO: 1;
TCR beta chains comprising constant regions which are selected from the group of TCR beta constant regions which comprise the amino acid sequence of SEQ ID NO: 2; or
TCR beta chains comprising constant regions which are selected from the group of TCR beta constant regions which comprise the amino acid sequence of SEQ ID NO: 15.

In some embodiments the TCR alpha chains comprise constant regions are selected from the group of TCR alpha constant regions which are at least about 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and the TCR beta chains comprise constant regions are selected from the group of TCR beta constant regions which are at least about 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 2.

Also encompassed is s method of preparing a library of synthetic polynucleotides encoding a plurality of TCR alpha chain constant regions and TCR beta chain constant regions, the method comprising:
Providing sequences of polynucleotides
   (i) encoding TCR alpha chain constant regions and TCR beta chain constant regions as defined herein; or
   (ii) encoding the TCR alpha chain and TCR beta chain as defined herein;
Assembling the synthetic polynucleotide sequences to produce a library of synthetic polynucleotides.

A further aspect refers to a kit comprising one or more of the libraries described herein.

Another aspect of the invention refers to nucleotide sequences of the synthetic polynucleotides defined herein in computer readable format. Accordingly, the invention also encompasses the amino acid sequences as defined herein in computer readable format.

“Nucleic acid” generally means a polymer of DNA or RNA, which can be single-stranded or double-stranded, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. The nucleic acid may be made synthetically, e.g. using art-recognized nucleic acid chemistry or enzymatically using, e.g. a polymerase. Preferably, the nucleic acids described herein are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication. The nucleic acids can be constructed based on chemical synthesis and/or enzymatic ligation reactions using procedures known in the art or commercially available (e.g. from Genscript, Thermo Fisher and similar companies). See, for example Sambrook et al., a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). The nucleic acid can comprise any nucleotide sequence which encodes any of the recombinant TCRs, polypeptides, or proteins, or functional portions or functional variants thereof.

The nucleic acid encoding the TCR may be modified. Useful modifications in the overall nucleic acid sequence may be codon optimization. Alterations may be made which lead to conservative substitutions within the expressed amino acid sequence. These variations can be made in complementarity determining and non-complementarity determining regions of the amino acid sequence of the TCR chain that do not affect function. Usually, additions and deletions should not be performed in the CDR3 region. Further, the modifications should not affect the base pairing. Moreover, as modifications in the constant regions that might impact the pairing of the TCR chains, changes beyond the variations indicated herein for the specific positions 14, 66, 92, 93, 107, 115, 118 and 119 (X01-X08 of FIG. 1A) of the TCR alpha chain constant region of SEQ ID NO: 1 and positions 5, 18, 22, 34, 157 and 164 (X09 to X14 of FIG. 1B) TCR beta chain constant region of SEQ ID NO: 2 (and the corresponding positions in SEQ ID NO: 15 and 16) should be avoided when modifying.

The terms “recombinant” and “recombinant TCR” as used in the present application refers to TCRs that have been introduced by any of the genetic engineering techniques into the T cells. The “recombinant TCR” also termed “exogenous TCR” may be engineered or may be a naturally occurring TCR which has a desired antigen specificity and which was isolated. Typically, these recombinant TCRs which were not endogenous to the T cell population, i.e. were not naturally expressed in the T cell population, i.e. were not expressed in the T cell population before transfer of the recombinant TCR.

Specific embodiments refer to a library of TCRs specific for NY-ESO-1. The experimental data shows beneficial effects for the TCRs specific for NY-ESO-1/LAGE-1 peptide having the sequence SLLMWITQC (SEQ ID NO: 33).

NY-ESO-1/LAGE-1 (also referred herein as “NY-ESO-1) belongs to the group of so called Cancer/Testis antigens. Cancer/Testis antigens are expressed in various malignant tumors and germ cells but in no other adult tissues. Therefore, NY-ESO-1/LAGE-1 is an interesting immunotherapeutic target antigen.

Accordingly, one embodiment refers to a library of synthetic polynucleotides encoding TCR alpha chains and TCR beta chains of a TCR capable of binding to a NY-ESO-1/LAGE-1 peptide having the sequence SLLMWITQC (SEQ ID NO: 33), comprising
Polynucleotides encoding TCR alpha chains comprising:
a) TCR alpha chain constant regions which are selected from the group of TCR alpha constant regions which are at least about 80% identical to the amino acid sequence as set out in SEQ ID NO: 1; wherein
   the amino acid at position 14 is selected from the group consisting of S, V, W, A, C, F, G, I, L, M and P;
   the amino acid at position 66 is selected from the group consisting of N, Q, S, T and Y;
   the amino acid at position 92 is selected from the group consisting of S, V, W, A C, F, G, I, L, M and P;
   the amino acid at position 93 is selected from the group consisting of S, V, W, A C, F, G, I, L, M and P;
   the amino acid at position 107 is selected from the group consisting of T, V, W, A, C, F, G, I, L, M and P;
   the amino acid at position115 is selected from the group consisting of S, V, W, A, C, F, G, I, L, M and P;
   the amino acid at position 118 is selected from the group consisting of G, I, L, M, P, V, W, A, C and F; and
   the amino acid at position 119 is selected from the group consisting of F, G, I, L, M, P, V, W, A and C;
b) TCR alpha chain variable region comprising a CDR1 having the amino acid sequence of SEQ ID NO: 34, a CDR2 having the amino acid sequence of SEQ ID NO: 35 and a CDR3 having the amino acid sequence of SEQ ID NO: 36;
Polynucleotides encoding TCR beta chains comprising
a) TCR beta chain constant regions which are selected from the group of TCR beta constant regions which are at least about 80% identical to the amino acid sequence as set out in SEQ ID NO: 2;
   wherein
   X at position 4 is selected from the group consisting of K, N;
   X at position 5 is selected from the group consisting of N, R, D, E, H and K;
   X at position 18 is selected from the group consisting of E, H, K, R and D;
   X at position 22 is selected from the group consisting of S, V, W, A, C, F, G, I, L, M and P;
   X at position 34 is selected from the group consisting of T, D, E, H, K, N and R;
   X at position 37 is selected from the group consisting of Y, F;
   X at position 136 is selected from the group consisting of E, V;
   X at position 157 is selected from the group consisting of T, V, W, A, C, F, G, I, L, M and P; and
   X at position 164 is selected from the group consisting of S, V, W, A, C, F, G, I, L, M and P;
   X at position 177 is selected from the group consisting of S, F;
   X at position 178 is R or deleted;
   X at position 179 is G or deleted;
b) TCR beta chain variable region comprising a CDR1 having the amino acid sequence of SEQ ID NO: 37, a CDR2 having the amino acid sequence of SEQ ID NO: 38 and a CDR3 having the amino acid sequence of SEQ ID NO: 39.

More specifically, one embodiment refers to a library of synthetic polynucleotides encoding TCR alpha chains and TCR beta chains of a TCR capable of binding to a NY-ESO-1/LAGE-1 peptide having the sequence SLLMWITQC (SEQ ID NO: 33), comprising
Polynucleotides encoding TCR alpha chains comprising:
a) TCR alpha chain constant regions which are selected from the group of TCR alpha constant regions which are at least about 80% identical to the amino acid sequence as set out in SEQ ID NO: 1;
   wherein
   the amino acid at position 14 is selected from the group consisting of S, V, W, A, C, F, G, I, L, M and P;
   the amino acid at position 66 is selected from the group consisting of N, Q, S, T and Y;
   the amino acid at position 92 is selected from the group consisting of S, V, W, A C, F, G, I, L, M and P;
   the amino acid at position 93 is selected from the group consisting of S, V, W, A C, F, G, I, L, M and P;
   the amino acid at position 107 is selected from the group consisting of T, V, W, A, C, F, G, I, L, M and P;
   the amino acid at position115 is selected from the group consisting of S, V, W, A, C, F, G, I, L, M and P;
   the amino acid at position 118 is selected from the group consisting of G, I, L, M, P, V, W, A, C and F; and
   the amino acid at position 119 is selected from the group consisting of F, G, I, L, M, P, V, W, A and C;
b) TCR alpha chain variable region comprising a CDR1 having the amino acid sequence of SEQ ID NO: 34, a CDR2 having the amino acid sequence of SEQ ID NO: 35 and a CDR3 having the amino acid sequence of SEQ ID NO: 36;
Polynucleotides encoding TCR beta chains comprising
a) TCR beta chain constant regions which are selected from the group of TCR beta constant regions which are at least about 80% identical to the amino acid sequence as set out in SEQ ID NO: 15; wherein
the amino acid at position 5 is selected from the group consisting of N, R, D, E, H and K;
the amino acid at position 18 is selected from the group consisting of E, H, K, R and D;
the amino acid at position 22 is selected from the group consisting of S, V, W, A, C, F, G, I, L, M and P;
the amino acid at position 34 is selected from the group consisting of T, D, E, H, K, N and R;
the amino acid at position 157 is selected from the group consisting of T, V, W, A, C, F, G, I, L, M and P; and
the amino acid at position 164 is selected from the group consisting of S, V, W, A, C, F, G, I, L, M and P;
   or
TCR beta chain constant regions which are selected from the group of TCR beta constant regions which are at least about 80% identical to the amino acid sequence as set out in SEQ ID NO: 16
wherein
the amino acid at position 5 is selected from the group consisting of N, R, D, E, H and K;
the amino acid at position 18 is selected from the group consisting of E, H, K, R and D;
the amino acid at position 22 is selected from the group consisting of S, V, W, A, C, F, G, I, L, M and P;
the amino acid at position 34 is selected from the group consisting of T, D, E, H, K, N and R;
the amino acid at position 157 is selected from the group consisting of T, V, W, A, C, F, G, I, L, M and P; and
the amino acid at position 164 is selected from the group consisting of S, V, W, A, C, F, G, I, L, M and P;
b) TCR beta chain variable region comprising a CDR1 having the amino acid sequence of SEQ ID NO: 37, a CDR2 having the amino acid sequence of SEQ ID NO: 38 and a CDR3 having the amino acid sequence of SEQ ID NO: 39.

Typically, the TCR specifically recognizes the amino acid sequence of SEQ ID NO: 33, which is presented by a molecule encoded by an HLA-A*02 gene.

Accordingly, also encompassed is a library of vectors comprising the library of synthetic polynucleotides encoding TCR alpha chains and TCR beta chains of a TCR capable of binding to a NY-ESO-1/LAGE-1 peptide having the sequence SLLMWITQC (SEQ ID NO: 33) as described above.

Further, some embodiments relate to a library of cells expressing the polynucleotides encoding TCR alpha chains and TCR beta chains of a TCR capable of binding to a NY-ESO-1/LAGE-1 peptide having the sequence SLLMWITQC (SEQ ID NO: 33) as described above.

One embodiment refers to a library of polypeptides encoding TCR alpha chains and TCR beta chains of a TCR capable of binding to a NY-ESO-1/LAGE-1 peptide having the sequence SLLMWITQC (SEQ ID NO: 33) as described above.

The use of said libraries for identifying a TCR receptor with enhanced TCR alpha and TRC beta chain pairing is also envisaged. According methods for isolating a library derived TCR capable of binding to a NY-ESO-1/LAGE-1 peptide having the sequence SLLMWITQC (SEQ ID NO: 33) with enhanced TCR alpha and beta chain pairing is also encompassed.

Accordingly, some embodiments relate to a TCR capable of binding to a NY-ESO-1/LAGE-1 peptide having the sequence SLLMWITQC (SEQ ID NO: 33) isolated from the TCR library as described above.

One embodiment refers a library of TCR alpha chains and TCR beta chains, comprising
TCR alpha chains comprising:
a) TCR alpha chain constant regions which are selected from the group of TCR alpha constant regions which are at least about 80% identical to the amino acid sequence as set out in SEQ ID NO 1:
   wherein
   the amino acid at position 14 is selected from the group consisting of S, V, W, A, C, F, G, I, L, M and P;
   the amino acid at position 66 is selected from the group consisting of N, Q, S, T and Y;
   the amino acid at position 92 is selected from the group consisting of S, V, W, A C, F, G, I, L, M and P;
   the amino acid at position 93 is selected from the group consisting of S, V, W, A C, F, G, I, L, M and P;
   the amino acid at position 107 is selected from the group consisting of T, V, W, A, C, F, G, I, L, M and P;
   the amino acid at position115 is selected from the group consisting of S, V, W, A, C, F, G, I, L, M and P;
   the amino acid at position 118 is selected from the group consisting of G, I, L, M, P, V, W, A, C and F; and
   the amino acid at position 119 is selected from the group consisting of F, G, I, L, M, P, V, W, A and C;
b) TCR alpha chain variable region comprising a CDR1 having the amino acid sequence of SEQ ID NO: 34, a CDR2 having the amino acid sequence of SEQ ID NO: 35 and a CDR3 having the amino acid sequence of SEQ ID NO: 36;
TCR beta chains comprising
a) TCR beta chain constant regions comprising constant regions which are selected from the group of TCR beta constant regions which are at least about 80% identical to the amino acid sequence as set out in SEQ ID NO: 15,
   wherein
   the amino acid at position 5 is selected from the group consisting of N, R, D, E, H and K;
   the amino acid at position 18 is selected from the group consisting of E, H, K, R and D;
   the amino acid at position 22 is selected from the group consisting of S, V, W, A, C, F, G, I, L, M and P;
   the amino acid at position 34 is selected from the group consisting of T, D, E, H, K, N and R;
   the amino acid at position 157 is selected from the group consisting of T, V, W, A, C, F, G, I, L, M and P; and
   the amino acid at position 164 is selected from the group consisting of S, V, W, A, C, F, G, I, L, M and P; or
TCR beta chain constant regions which are selected from the group of TCR beta constant regions which are at least about 80% identical to the amino acid sequence as set out in SEQ ID NO 16;
   wherein
   the amino acid at position 5 is selected from the group consisting of N, R, D, E, H and K;
   the amino acid at position 18 is selected from the group consisting of E, H, K, R and D;
   the amino acid at position 22 is selected from the group consisting of S, V, W, A, C, F, G, I, L, M and P;
   the amino acid at position 34 is selected from the group consisting of T, D, E, H, K, N and R;
   the amino acid at position 157 is selected from the group consisting of T, V, W, A, C, F, G, I, L, M and P; and
      X at position 164 is selected from the group consisting of S, V, W, A, C, F, G, I, L, M and P
b) TCR beta chain variable region comprising a CDR1 having the amino acid sequence of SEQ ID NO: 37, a CDR2 having the amino acid sequence of SEQ ID NO: 38 and a CDR3 having the amino acid sequence of SEQ ID NO: 39.

Further embodiments refer to a TCR capable of binding to a NY-ESO-1/LAGE-1 peptide having the sequence SLLMWITQC (SEQ ID NO: 33), wherein the TCR comprises:
a TCR alpha chain comprising
a) TCR alpha chain variable region comprising a CDR1 having the amino acid sequence of SEQ ID NO: 34, a CDR2 having the amino acid sequence of
SEQ ID NO: 35 and a CDR3 having the amino acid sequence of SEQ ID NO: 36;
b) a constant TCR alpha region comprising the sequence selected from the group of constant TCR alpha regions represented by the sequence as set in SEQ ID NO 1:
a TCR beta chain comprising
a) TCR beta chain variable region comprising a CDR1 having the amino acid sequence of SEQ ID NO: 37, a CDR2 having the amino acid sequence of SEQ ID NO: 38 and a CDR3 having the amino acid sequence of SEQ ID NO: 39;
b) a constant TCR beta region comprising the sequence selected from the group of constant TCR beta regions represented by the sequence as set in SEQ ID NO: 15, or
   a constant TCR beta region comprising the sequence selected from the group of constant TCR beta regions represented by the sequence as set in SEQ ID NO: 16.

The examples support that the TCRs of the invention show elevated expression levels of the heterologous TCR compared to wild type and/or elevated activation levels compared to wild type.

A specific embodiment refers to a TCR capable of binding to a NY-ESO-1/LAGE-1 peptide having the sequence SLLMWITQC (SEQ ID NO: 33), wherein the TCR comprises:
A TCR alpha chain comprising
a) a variable TCR alpha chain region comprising a CDR1 having the amino acid sequence of SEQ ID NO: 34, a CDR2 having the amino acid sequence of SEQ ID NO: 34 and a CDR3 having the amino acid sequence of SEQ ID NO: 35;
b) A constant TCR alpha region comprising the sequence SEQ ID NO: 12
A TCR beta chain comprising
a) a variable TCR beta chain region comprising a CDR1 having the amino acid sequence of SEQ ID NO: 39, a CDR2 having the amino acid sequence of SEQ ID NO: 40 and a CDR3 having the amino acid sequence of SEQ ID NO: 41;
b) a constant TCR beta region comprising the sequence SEQ ID NO: 13;

Typically, the TCR specifically recognizes the amino acid sequence of SEQ ID NO: 33, which is presented by a molecule encoded by an HLA-A*02 gene.

In one embodiment the TCR comprises
a) a variable TCR α region having an amino acid sequence which is at least 80% identical to SEQ ID NO: 40 and b) a variable TCR β region having an amino acid sequence which is at least 80% identical to SEQ ID NO: 41.

In one embodiment, the TCR comprises
a) a variable TCR α region having the amino acid sequence of SEQ ID NO: 40 and
b) a variable TCR β region having the amino acid sequence of SEQ ID NO: 41.

Some embodiments refer to a nucleic acid encoding the TCR or the polypeptide as described above as well as vector containing such nucleic acid.

Accordingly some embodiments refer to a cell expressing said TCR capable of binding to a NY-ESO-1/LAGE-1 peptide having the sequence SLLMWITQC (SEQ ID NO: 33), as described above.

The skilled person understands that the general definitions and embodiments as disclosed herein also apply to the disclosure and aspects with regard to TCRs capable of binding to a NY-ESO-1/LAGE-1 peptide having the sequence SLLMWITQC (SEQ ID NO: 33) and corresponding libraries.

### Examples

### Library Design

“TCR-C-enhancement library” is a DNA library carrying 10¹¹ TCR α and β constant region variants of the PRAME_{VLD}-TCR and was generated using TRIM technology. TCR constant regions contain six mutation sites in the TCR C-β chain and eight mutation sites in the TCR C-α chain (FIGS. 1A and B). Amino acid substitutions were chosen based on a broad literature evaluation for residues that are important for: improving TCR signal transduction through better TCR interaction with CD3 subunits; post-translational modifications that might be detrimental for TCR avidity (e.g N-linked glycosylated positions); binding to lipid rafts resulting in better clustering [Cohen et. al., Cancer Res. 2007; Kuball et. al. J Exp Med, 2009; Haga-Friedman et. al. J Immunol, 2012; Bethune et. al. eLife 2016;]. Additionally, amino acid positions in murine constant regions that have been proved to be important for TCR avidity and could potentially influence pairing of alpha and beta chains were also chosen for diversification [Sommermayer and Uckert J Immunol, 2010]. Library diversification was done taking into consideration that some positions might be risky to change thus those positions had a higher frequency of wt-derived amino acid sequences. Also not all 20 amino acids could be used for the diversification at each position as the complexity of the library would be too high and could not be reasonably synthesized.

The sequences coding for alpha and beta chains of PRAME_{VLD}-TCR are separated with a linker and P2A sequence coding for self-cleaving peptide (FIG. 1C). Library contains also a mTag BFP sequence preceded with a linker and P2A sequence. The library was cloned into a retroviral vector that allows transduction of the library into recipient T cells with the aim of finding optimal TCR mutation combinations for increased functional avidity.

### Screening Procedure

Retroviral supernatants of the library as well as PRAME_{VLD}-WT-TCR construct, which differed from library PRAME_{VLD}-TCR amino acid sequence only in regard to the mutation sites in the constant α and β chains (FIGS. 1A and B), were produced in HEK293FT cells. Viral supernatants were titrated and diluted to achieve multiplicity of infection (MOI) equal or lower than 1, thus maximizing the number of cells with a single integration of a viral particle. Additionally, a TCR signal reporter system, which allows high-throughput flow cytometry-based screening for exogenously introduced and activated TCRs, was generated. For that purpose, Jurkat 76 T cell line without endogenous expression of TCR, but expressing CD8, was engineered to express eGFP under the control of a nuclear factor of activated T cells (NFAT) response element (Jurkat-ieGFP Biosensors). These cells were used for transduction, followed by bulk fluorescence activated cell sorting for BFP positive cells (transduced cells) (FIG. 2). In the next step, transduced cells were co-cultured with VLDGLDVLL (VLD) peptide loaded T2 cells or SK-MEL23 melanoma cell line expressing PRAME antigen, both expressing also the HLA of interest, and after 24h a single cell sort for CD3⁺eGFP^{high} cells was performed. CD3 can be only detected on the cell surface if it is associated with an alpha and beta TCR heterodimer, thus cells without a paired alpha beta TCR will be CD3 negative and the amount of CD3 will be directly related to the amount of the alpha-beta paired chains of the TCR. Therefore CD3 can be used as selection marker. Based on CD3 positivity, Jurkat-ieGFP Biosensor cells successfully expressing TCR were selected. Additionally, through gating on cells with high expression of eGFP, only cells transduced with highly functional TCRs were isolated. Single cell clones were expanded to the numbers allowing further testing for reactivity to T2 cells loaded with 10⁻⁵ M concentration of VLD peptide or tumor cell lines with differential expression of PRAME antigen. PRAME_{VLD}-WT-TCR-Jurkat-ieGFP Biosensors (WT) were used as a control throughout the whole screening process. Based on the superior functionality compared to the WT-TCR, one promising clone caring TCRs from the library was selected.

### Characterization of the Selected Clone

For the further characterization of the selected clone (called 8-9), derived from library screening, the level of TCR expression on the surface of the library clone and WT was analyzed using flow cytometry. Library clone 8-9 showed highly elevated levels of TCR expression compared to the WT (FIGS. 3A and B), that may account for its increased functionality observed in the screening procedure.

Additionally, response to the irrelevant peptide derived from the same PRAME antigen was investigated to ensure high specificity of the TCRs expressed on the surface of the selected clone (FIGS. 4A and B). For this purpose, T2 cells expressing the HLA of interest were loaded with 10⁻⁵ M concentration of relevant (VLD) or irrelevant SLLQHLIGL (SLL) peptide and co-cultured with the library clone 8-9 or the WT cells. After 24h of co-culture, eGFP signal induced upon TCR activation was measured with flow cytometry. The specific activation could only be seen when the library clone or WT were co-cultured with T2 cells loaded with relevant peptide, thereby confirming the peptide specificity of the selected clone.

To test whether the selected library clone can recognize and respond to the antigen naturally processed and presented on the cell surface of tumor cells, its reactivity to tumor cell line SK-MEL23 expressing PRAME antigen (1150.3 reads per kilobase million [RPKM], data not shown) was investigated (FIG. 5).

As a result of 24h co-culture with the SK-MEL23 cell line, the library clone showed increased expression of inducible eGFP levels in comparison to WT measured by flow cytometry. The selected library clone was shown to carry highly functional TCRs, which are able to recognize naturally processed and presented peptide more efficiently than WT.

### Reconstitution of 8-9 Clone Derived TCR and Validation

As a next step, TCR sequence derived from clone 8-9 (called 8-9 TCR) and WT-TCR sequence, which differed from 8-9 TCR nucleotide sequence only in regard to the mutation sites in the constant α and β chains introduced through the library, were reconstituted in Jurkat-ieGFP Biosensor cells or in CD8+ T cells derived from three healthy donors. Reconstitution was performed with retroviral transduction at high MOI. With comparable expression of BFP (transduction efficiency marker), TCR expression levels were highly increased in 8-9 TCR transduced Jurkat-ieGFP Biosensor cells or CD8+ T cells from all three healthy donors compared to the same cell types transduced with WT-TCR (FIG. 6).

The expression of TCR 8-9 compared to the WT-TCR is higher in both, Jurkat-ieGFP cells and CD8+ T cells (FIG. 6). The difference between WT-TCR transduced and 8-9-TCR transduced CD8+ T cells from healthy donors is significantly smaller compared with the difference of WT-TCR transduced and 8-9-TCR transduced ieGFP Jurkat cells. Nevertheless, the reactivity of the 8-9 TCR transduced CD8+ T cells is markedly higher than that of the WT-TCR transduced T cells, comparable to the results seen in ieGFP-Jurkat cells (FIG. 7). This supports that the TCRs described herein identified by the method of the invention not only show an increased surface expression but also a reduced mispairing with the endogenous TCR chains compared to WT-TCR.

8-9 TCR or WT-TCR-transduced Jurkat-ieGFP Biosensor cells were co-cultured with T2 cells loaded with titrated concentrations of VLD peptide ranging from 10-10 to 10⁻⁵ M. After 20h of co-culture, percentage of eGFP positive cells as well as geometric mean fluorescence intensity of eGFP signal on the BFP positive cells (transduced cells) was assessed with flow cytometry. In both read-outs 8-9 TCR-transduced cells showed higher signals when compared to the WT-TCR-transduced cells (FIG. 7A).

In the similar way, 8-9 TCR or WT-TCR-transduced CD8+ T cells were co-cultured with T2 cells loaded with titrated VLD peptide concentrations. After 24h, supernatants were collected and IFNγ ELISA was performed. IFNγ levels secreted by CD8+ T cells transduced with the 8-9 TCR were much higher than for T cells carrying the WT-TCR construct (in the 10⁻⁹ to 10⁻⁵ peptide concentration range) supporting the results derived from Jurkat-ieGFP Biosensor co-cultures (FIG. 7B).

Transfer of Constant Region to TCR with Different Specificity

To further characterize the potency of the identified PP constant sequence, i.e. alpha chain constant region SEQ ID NO: 12 and beta chain constant region SEQ ID NO 13, was transferred to a TCR with different specificity, the NY-ESO variable regions (NY-ESO-mut_4.3 Cb1, SEQ ID NO: 47 and SEQ ID NO: 45). The NY-ESO-mut_4.3 Cb1 TCR equipped T cells were compared to T cells either equipped with the wildtype TCR or the minimal murinized TCR (NY-ESO_mmCb1 SEQ ID NO: 48 and SEQ ID NO: 49) when co-cultured with tumor cell lines (FIG. 8). This experiment shows, that the beneficial effect of the PP mutations identified in VLD-TCRs can be transferred to TCRs with other specificities (here NY-ESO). The PP-NY-ESO-TCR T cells show increased IFN-γ release compared to the wildtype TCR upon co-culturing with target cells (Mel624.38). The activity is comparable to the minimal murinized version of the NY-ESO TCR T cells.

Additionally, it was tested, if the mutations can be transferred to the second beta constant chain allele (Cb2) (FIG. 9 A+B), together with the identified PP alpha chain constant region SEQ ID NO: 12, resulting in the TCR named NY-ESO-mut_4.3 Cb2 (SEQ ID NO: 46 and SEQ ID NO: 47). Therefore, the PP mutations were used at the same positions in Cb2 as originally identified in Cb1. FIG. 9A shows the comparable expression of NY-ESO-mut_4.3 Cb2 TCR (MFI 2179, transduction efficiency 73%) compared to wildtype NY-ESO TCR (MFI 1883, transduction efficiency 69.7%). The NY-ESO-mut_4.3_Cb2 TCR T cells show slightly better IFN-γ expression upon co-culturing with target cells (MOLP-2) compared to wildtype TCR T cells. The activity is comparable to the minimal murinized TCR NY-ESO_mmCb2 (SEQ ID NO: 48 and SEQ ID NO: 50) T cells, thereby proving that the PP mutations can be transferred to the second beta constant chain allele Cb2.

Newly identified precision pairing TCR sequences

### Further Sequences of the Library Show Improved Effect

Using the PP library and the already described method, additional sequences were identified that showed an increase activity compared to wt sequence using the Jurkat biosensor system (FIG. 10A-D).

Several clones and WT were co-cultured with T2 cells loaded with either relevant VLDGLDVLL peptide, irrelevant peptide, or left alone (T cells only). After 24h of co-culture, cells were collected and percentage of eGFP positive cells and gMFI of eGFP signal on the transduced effector cells was measured with flow cytometry, gMFI of eGFP channel was plotted.

For analyzing expression of precision paired TCRs in TCR-deficient Jurkat-76 CD8+ieGFP cells, transduced Jurkat-76 cells were analyzed for TCR expression by staining with anti TCR Vβ1 (TRBV9) antibody [PE, clone BL37.2, Beckman Coulter] antibody and subsequent flow cytometric analysis.

ppTCR clones showed equal or higher expression of the TCR on the cell surface of Jurkat 76−/−CD8+ieGFP cells compared to wildtype TCR transduce Jurkat 76−/− CD8+ieGFP cells (FIG. 10E). In a co-culture experiment all identified PP clones showed better reactivity compared to the wildtype clones, depicted by either gMFI or % GFP (FIG. 10 A-D). This shows that the precision pairing library allows identification of several TCR constant chain variants that show superiority over the wildtype TCR constant chain sequences.

The application further comprises the following items:
Item 1: A library of synthetic polynucleotides encoding TCR alpha chain constant regions and TCR beta chain constant regions, wherein the library comprises:
   Polynucleotides encoding TCR alpha chain constant regions which are selected from the group of TCR alpha constant regions which are at least about 80% identical to the amino acid sequence as set out in SEQ ID NO: 1;
   Polynucleotides encoding TCR beta chain constant regions which are selected from the group of TCR beta constant regions which are at least about 80% identical to the amino acid sequence as set out in SEQ ID NO: 2.
Item 2: A library of synthetic polynucleotides encoding TCR alpha chain constant regions and TCR beta chain constant regions, wherein the library comprises:
   Polynucleotides encoding TCR alpha chain constant regions which are selected from the group of TCR alpha constant regions which are at least about 80% identical to the amino acid sequence as set out
   in SEQ ID NO: 1;
      wherein
      the amino acid at position 14 is selected from the group consisting of S, V, W, A, C, F, G, I, L, M and P;
      the amino acid at position 66 is selected from the group consisting of N, Q, S, T and Y;
      the amino acid at position 92 is selected from the group consisting of S, V, W, A C, F, G, I, L, M and P;
      the amino acid at position 93 is selected from the group consisting of S, V, W, A C, F, G, I, L, M and P;
      the amino acid at position 107 is selected from the group consisting of T, V, W, A, C, F, G, I, L, M and P;
      the amino acid at position115 is selected from the group consisting of S, V, W, A, C, F, G, I, L, M and P;
      the amino acid at position 118 is selected from the group consisting of G, I, L, M, P, V, W, A, C and F; and
      the amino acid at position 119 is selected from the group consisting of F, G, I, L, M, P, V, W, A and C;
   Polynucleotides encoding TCR beta chain constant regions which are selected from the group of TCR beta constant regions which are at least about 80% identical to the amino acid sequence as set out in SEQ ID NO: 2, wherein
      the amino acid at position 4 is selected from the group consisting of K, N;
      the amino acid at position 5 is selected from the group consisting of N, R, D, E, H and K;
      the amino acid at position 18 is selected from the group consisting of E, H, K, R and D;
      the amino acid at position 22 is selected from the group consisting of S, V, W, A, C, F, G, I, L, M and P;
      the amino acid at position 34 is selected from the group consisting of T, D, E, H, K, N and R;
      the amino acid at position 37 is selected from the group consisting of Y, F;
      the amino acid at position 136 is selected from the group consisting of E, V;
      the amino acid at position 157 is selected from the group consisting of T, V, W, A, C, F, G, I, L, M and P; and
      the amino acid at position 164 is selected from the group consisting of S, V, W, A, C, F, G, I, L, M and P;
      the amino acid at position 177 is selected from the group consisting of S, F;
      the amino acid at position 178 is R or deleted;
      the amino acid at position 179 is G or deleted.
Item 3: A library of synthetic polynucleotides encoding TCR alpha chain constant regions and TCR beta chain constant regions, wherein the library comprises:
   Polynucleotides encoding TCR alpha chain constant regions which are selected from the group of TCR alpha constant regions which are at least about 80% identical to the amino acid sequence as set out in SEQ ID NO: 1;
   1 Polynucleotides encoding TCR beta chain constant regions which are selected from the group of TCR beta constant regions which are at least about 80% identical to the amino acid sequence as set out in SEQ ID NO: 15; or
   Polynucleotides encoding TCR beta chain constant regions which are selected from the group of TCR beta constant regions which are at least about 80% identical to the amino acid sequence as set out in SEQ ID NO: 16.
Item 4: A library of synthetic polynucleotides encoding TCR alpha chains and TCR beta chains, comprising
   Polynucleotides encoding TCR alpha chains comprising constant regions which are selected from the group of TCR alpha constant regions which are at least about 80% identical to the amino acid sequence as set out in SEQ ID NO: 1;
      wherein
      the amino acid at position 14 is selected from the group consisting of S, V, W, A, C, F, G, I, L, M and P;
      the amino acid at position 66 is selected from the group consisting of N, Q, S, T and Y;
      the amino acid at position 92 is selected from the group consisting of S, V, W, A C, F, G, I, L, M and P;
      the amino acid at position 93 is selected from the group consisting of S, V, W, A C, F, G, I, L, M and P;
      the amino acid at position 107 is selected from the group consisting of T, V, W, A, C, F, G, I, L, M and P;
      the amino acid at position115 is selected from the group consisting of S, V, W, A, C, F, G, I, L, M and P;
      the amino acid at position 118 is selected from the group consisting of G, I, L, M, P, V, W, A, C and F; and
   the amino acid at position 119 is selected from the group consisting of F, G, I, L, M, P, V, W, A and C;
   Polynucleotides encoding TCR beta chains comprising constant regions which are selected from the group of TCR beta constant regions which are at least about 80% identical to the amino acid sequence as set out in SEQ ID NO: 2
      wherein
      the amino acid at position 4 is selected from the group consisting of K, N;
      the amino acid at position 5 is selected from the group consisting of N, R, D, E, H and K;
      the amino acid at position 18 is selected from the group consisting of E, H, K, R and D;
      the amino acid at position 22 is selected from the group consisting of S, V, W, A, C, F, G, I, L, M and P;
      the amino acid at position 34 is selected from the group consisting of T, D, E, H, K, N and R;
      the amino acid at position 37 is selected from the group consisting of Y, F;
      the amino acid at position 136 is selected from the group consisting of E, V;
      the amino acid at position 157 is selected from the group consisting of T, V, W, A, C, F, G, I, L, M and P; and
      the amino acid at position 164 is selected from the group consisting of S, V, W, A, C, F, G, I, L, M and P;
      the amino acid at position 177 is selected from the group consisting of S, F;
      the amino acid at position 178 is R or deleted;
      the amino acid at position 179 is G or deleted.
Item 5: A library of synthetic polynucleotides encoding TCR alpha chains and TCR beta chains, comprising
Polynucleotides encoding TCR alpha chains comprising constant regions which are selected from the group of TCR alpha constant regions which are at least about 80% identical to the amino acid sequence as set out in SEQ ID NO: 1;
Polynucleotides encoding TCR beta chains comprising constant regions which are selected from the group of TCR beta constant regions which are at least about 80% identical to the amino acid sequence as set out in SEQ ID NO: 15
   wherein
   the amino acid at position 5 is selected from the group consisting of N, R, D, E, H and K;
   the amino acid at position 18 is selected from the group consisting of E, H, K, R and D;
   the amino acid at position 22 is selected from the group consisting of S, V, W, A, C, F, G, I, L, M and P;
   the amino acid at position 34 is selected from the group consisting of T, D, E, H, K, N and R;
   the amino acid at position 157 is selected from the group consisting of T, V, W, A, C, F, G, I, L, M and P; and
   the amino acid at position 164 is selected from the group consisting of S, V, W, A, C, F, G, I, L, M and P;
   or
   Polynucleotides encoding TCR beta chains comprising constant regions which are selected from the group of TCR beta constant regions which are at least about 80% identical to the amino acid sequence as set out in SEQ ID NO: 16
   wherein
   the amino acid at position 5 is selected from the group consisting of N, R, D, E, H and K;
   the amino acid at position 18 is selected from the group consisting of E, H, K, R and D;
   the amino acid at position 22 is selected from the group consisting of S, V, W, A, C, F, G, I, L, M and P;
   the amino acid at position 34 is selected from the group consisting of T, D, E, H, K, N and R;
   the amino acid at position 157 is selected from the group consisting of T, V, W, A, C, F, G, I, L, M and P; and
   the amino acid at position 164 is selected from the group consisting of S, V, W, A, C, F, G, I, L, M and P.
Item 6: Library of synthetic polynucleotides according to any one of items 1 to 5, wherein the library comprises polynucleotides encoding a TCR alpha chain and a TCR beta chain, each polynucleotide comprising:
   Polynucleotide encoding TCR alpha chains as defined in item 2; and
   Polynucleotide encoding TCR beta chains as defined in item 2;
   Optionally a selection marker
   Optionally one or more ribosomal entry site or self-cleaving peptides of the 2A family.
Item 7: Library of synthetic polynucleotides according to item 6, wherein the selection marker is a fluorescent tag.
Item 8: Library of synthetic polynucleotides according to item 7, wherein the fluorescent tag is MTagBFP.
Item 9: Library of synthetic polynucleotides according to of items 1 to 8, wherein the library comprises at least 1×10⁵, preferably, 1×10¹⁰, more preferably 2.1×10¹¹, such as 2.25×10¹³ unique molecules.
Item 10: A library of vectors comprising the polynucleotide library according to any one of items 1 to 9.
Item 11: The library of vectors according to item 10, wherein the vectors are retroviral vectors.
Item12: A library of cells, comprising the library of vectors according to item 10 or 11.
Item 13: A library of cells expressing the polynucleotides defined in items 1 to 7.
Item 14: A library of polypeptides encoded by the library of synthetic polynucleotides according to items 1 to 9.
Item 15: Use of the library of items 1 to 14 for identifying a TCR receptor with enhanced TCR alpha and TCR beta chain pairing.
Item 16: A method for isolating a TCR with enhanced TCR alpha and beta chain pairing, comprising the steps:
   Co-culturing the library of cells according to items 12 and 13 with antigen presenting cells presenting the HLA bound form of the target antigen of the TCR
   Selecting cells showing elevated expression levels of the heterologous TCR compared to wild type
   Selecting cells showing elevated activation levels compared to wild type.
Item 17: A TCR isolated from the TCR library as described in item 16.
Item 18: A library of TCR alpha chains and TCR beta chains, comprising
   TCR alpha chains comprising constant regions which are selected from the group of TCR alpha constant regions which are at least about 80% identical to the amino acid sequence as set out in SEQ ID NO: 1;
      wherein
      the amino acid at position 14 is selected from the group consisting of S, V, W, A, C, F, G, I, L, M and P;
      the amino acid at position 66 is selected from the group consisting of N, Q, S, T and Y;
      the amino acid at position 92 is selected from the group consisting of S, V, W, A C, F, G, I, L, M and P;
      the amino acid at position 93 is selected from the group consisting of S, V, W, A C, F, G, I, L, M and P;
      the amino acid at position 107 is selected from the group consisting of T, V, W, A, C, F, G, I, L, M and P;
      the amino acid at position115 is selected from the group consisting of S, V, W, A, C, F, G, I, L, M and P;
      the amino acid at position 118 is selected from the group consisting of G, I, L, M, P, V, W, A, C and F; and
      the amino acid at position 119 is selected from the group consisting of F, G, I, L, M, P, V, W, A and C;
   TCR beta chains comprising constant regions which are selected from the group of TCR beta constant regions which are at least about 80% identical to the amino acid sequence as set out in SEQ ID NO: 2
      wherein
      the amino acid at position 4 is selected from the group consisting of K, N;
      the amino acid at position 5 is selected from the group consisting of N, R, D, E, H and K;
      the amino acid at position 18 is selected from the group consisting of E, H, K, R and D;
      the amino acid at position 22 is selected from the group consisting of S, V, W, A, C, F, G, I, L, M and P;
      the amino acid at position 34 is selected from the group consisting of T, D, E, H, K, N and R;
      the amino acid at position 37 is selected from the group consisting of Y, F;
      the amino acid at position 136 is selected from the group consisting of E, V;
      the amino acid at position 157 is selected from the group consisting of T, V, W, A, C, F, G, I, L, M and P; and
      the amino acid at position 164 is selected from the group consisting of S, V, W, A, C, F, G, I, L, M and P;
      the amino acid at position 177 is selected from the group consisting of S, F;
      the amino acid at position 178 is R or deleted;
      the amino acid at position 179 is G or deleted.
Item 19: A library of TCR alpha chains and TCR beta chains, comprising
   a) TCR alpha chains comprising constant regions which are selected from the group of TCR alpha constant regions which are at least about 80% identical to the amino acid sequence as set out in SEQ ID NO: 1;
      wherein
      the amino acid at position 14 is selected from the group consisting of S, V, W, A, C, F, G, I, L, M and P;
      the amino acid at position 66 is selected from the group consisting of N, Q, S, T and Y;
      the amino acid at position 92 is selected from the group consisting of S, V, W, A C, F, G, I, L, M and P;
      the amino acid at position 93 is selected from the group consisting of S, V, W, A C, F, G, I, L, M and P;
      the amino acid at position 107 is selected from the group consisting of T, V, W, A, C, F, G, I, L, M and P;
      the amino acid at position115 is selected from the group consisting of S, V, W, A, C, F, G, I, L, M and P;
      the amino acid at position 118 is selected from the group consisting of G, I,
      L, M, P, V, W, A, C and F; and
      the amino acid at position 119 is selected from the group consisting of F, G, I, L, M, P, V, W, A and C;
   b) TCR beta chains comprising constant regions which are selected from the group of TCR beta constant regions which are at least about 80% identical to the amino acid sequence as set out in SEQ ID NO: 15;
      wherein
      the amino acid at position 5 is selected from the group consisting of N, R, D, E, H and K;
      the amino acid at position 18 is selected from the group consisting of E, H, K, R and D;
      the amino acid at position 22 is selected from the group consisting of S, V, W, A, C, F, G, I, L, M and P;
      the amino acid at position 34 is selected from the group consisting of T, D, E, H, K, N and R;
      the amino acid at position 157 is selected from the group consisting of T, V, W, A, C, F, G, I, L, M and P; and
      the amino acid at position 164 is selected from the group consisting of S, V, W, A, C, F, G, I, L, M and P;
   or
      TCR beta chains comprising constant regions which are selected from the group of TCR beta constant regions which are at least about 80% identical to the amino acid sequence as set out in SEQ ID NO: 16
      wherein
      the amino acid at position 5 is selected from the group consisting of N, R, D, E, H and K;
      the amino acid at position 18 is selected from the group consisting of E, H, K, R and D;
      the amino acid at position 22 is selected from the group consisting of S, V, W, A, C, F, G, I, L, M and P;
      the amino acid at position 34 is selected from the group consisting of T, D, E, H, K, N and R;
      the amino acid at position 157 is selected from the group consisting of T, V, W, A, C, F, G, I, L, M and P; and
      the amino acid at position 164 is selected from the group consisting of S, V, W, A, C, F, G, I, L, M and P.
Item 20: A method of preparing a library of synthetic polynucleotides encoding a plurality of TCR alpha chain constant regions and TCR beta chain constant regions, the method comprising:
   Providing sequences of polynucleotides
      (i) encoding TCR alpha chain constant regions and TCR beta chain constant regions as defined in item 1; or
      (ii) encoding the TCR alpha chain and TCR beta chain as defined in item 2;
   Assembling the synthetic polynucleotide sequences to produce a library of synthetic polynucleotides.
Item 21: A kit comprising the library as defined in items 1 to 14.
Item 22: The nucleotide sequences of the synthetic polynucleotides defined in items 1 to 14 in computer readable format.
Item 23: The amino acid sequences defined in item 1 to 14 in computer readable format.
Item 24: A TCR, comprising
   A TCR alpha chain comprising constant region which is at least about 80% identical to the amino acid sequence as set out in SEQ ID NO: 1;
      wherein
      the amino acid at position 14 is selected from the group consisting of S, V, W, A, C, F, G, I, L, M and P;
      the amino acid at position 66 is selected from the group consisting of N, Q, S, T and Y;
      the amino acid at position 92 is selected from the group consisting of S, V, W, A C, F, G, I, L, M and P;
      the amino acid at position 93 is selected from the group consisting of S, V, W, A C, F, G, I, L, M and P;
      the amino acid at position 107 is selected from the group consisting of T, V, W, A, C, F, G, I, L, M and P;
      the amino acid at position115 is selected from the group consisting of S, V, W, A, C, F, G, I, L, M and P;
      the amino acid at position 118 is selected from the group consisting of G, I, L, M, P, V, W, A, C and F; and
      the amino acid at position 119 is selected from the group consisting of F, G, I, L, M, P, V, W, A and C;
   TCR beta chains comprising a constant regions which is at least about 80% identical to the amino acid sequence as set out in SEQ ID NO: 2;
   wherein
      the amino acid at position 4 is selected from the group consisting of K, N;
      the amino acid at position 5 is selected from the group consisting of N, R, D, E, H and K;
      the amino acid at position 18 is selected from the group consisting of E, H, K, R and D;
      the amino acid at position 22 is selected from the group consisting of S, V, W, A, C, F, G, I, L, M and P;
      the amino acid at position 34 is selected from the group consisting of T, D, E, H, K, N and R;
      the amino acid at position 37 is selected from the group consisting of Y, F;
      the amino acid at position 136 is selected from the group consisting of E, V;
      the amino acid at position 157 is selected from the group consisting of T, V, W, A, C, F, G, I, L, M and P; and
      the amino acid at position 164 is selected from the group consisting of S, V, W, A, C, F, G, I, L, M and P;
      the amino acid at position 177 is selected from the group consisting of S, F;
      the amino acid at position 178 is R or deleted;
      the amino acid at position 179 is G or deleted.
Item 25: A TCR, comprising
   A TCR alpha chain comprising constant region which is at least about 80% identical to the amino acid sequence as set out in SEQ ID NO: 1;
      wherein
      the amino acid at position 14 is selected from the group consisting of S, V, W, A, C, F, G, I, L, M and P;
      the amino acid at position 66 is selected from the group consisting of N, Q, S, T and Y;
      the amino acid at position 92 is selected from the group consisting of S, V, W, A C, F, G, I, L, M and P;
      the amino acid at position 93 is selected from the group consisting of S, V, W, A C, F, G, I, L, M and P;
      the amino acid at position 107 is selected from the group consisting of T, V, W, A, C, F, G, I, L, M and P;
      the amino acid at position115 is selected from the group consisting of S, V, W, A, C, F, G, I, L, M and P;
      the amino acid at position 118 is selected from the group consisting of G, I, L, M, P, V, W, A, C and F; and
      the amino acid at position 119 is selected from the group consisting of F, G, I, L, M, P, V, W, A and C;
   TCR beta chains comprising a constant regions which is at least about 80% identical to the amino acid sequence as set out in SEQ ID NO: 15; wherein X at position 5 is selected from the group consisting of N, R, D, E, H and K;
      the amino acid at position 18 is selected from the group consisting of E, H, K, R and D;
      the amino acid at position 22 is selected from the group consisting of S, V, W, A, C, F, G, I, L, M and P;
      the amino acid at position 34 is selected from the group consisting of T, D, E, H, K, N and R;
      the amino acid at position 157 is selected from the group consisting of T, V, W, A, C, F, G, I, L, M and P; and
      the amino acid at position 164 is selected from the group consisting of S, V, W, A, C, F, G, I, L, M and P; or
      TCR beta chains comprising a constant regions which is at least about 80% identical to the amino acid sequence as set out in SEQ ID NO: 16, wherein
      the amino acid at position 5 is selected from the group consisting of N, R, D, E, H and K;
      the amino acid at position 18 is selected from the group consisting of E, H, K, R and D;
      the amino acid at position 22 is selected from the group consisting of S, V, W, A, C, F, G, I, L, M and P;
      the amino acid at position 34 is selected from the group consisting of T, D, E, H, K, N and R;
      the amino acid at position 157 is selected from the group consisting of T, V, W, A, C, F, G, I, L, M and P; and
      the amino acid at position 164 is selected from the group consisting of S, V, W, A, C, F, G, I, L, M and P.
Item 26: A library of synthetic polynucleotides encoding TCR alpha chains and TCR beta chains of a TCR capable of binding to a NY-ESO-1/LAGE-1 peptide having the sequence SLLMWITQC (SEQ ID NO: 33), comprising
   Polynucleotides encoding TCR alpha chains comprising:
      a) TCR alpha chain constant regions which are selected from the group of TCR alpha constant regions which are at least about 80% identical to the amino acid sequence as set out in SEQ ID NO: 1;
      b) TCR alpha chain variable region comprising a CDR1 having the amino acid sequence of SEQ ID NO: 34, a CDR2 having the amino acid sequence of SEQ ID NO: 35 and a CDR3 having the amino acid sequence of SEQ ID NO: 36;
   Polynucleotides encoding TCR beta chains comprising
      a) TCR beta chain constant regions which are selected from the group of TCR beta constant regions which are at least about 80% identical to the amino acid sequence as set out in SEQ ID NO: 2;
      b) TCR beta chain variable region comprising a CDR1 having the amino acid sequence of SEQ ID NO: 37, a CDR2 having the amino acid sequence of SEQ ID NO: 38 and a CDR3 having the amino acid sequence of SEQ ID NO: 39.
Item 27: A library of synthetic polynucleotides encoding TCR alpha chains and TCR beta chains of a TCR capable of binding to a NY-ESO-1/LAGE-1 peptide having the sequence SLLMWITQC (SEQ ID NO: 33), comprising
   Polynucleotides encoding TCR alpha chains comprising:
      a) TCR alpha chain constant regions which are selected from the group of TCR alpha constant regions which are at least about 80% identical to the amino acid sequence as set out in SEQ ID NO: 1;
         wherein
         the amino acid at position 14 is selected from the group consisting of S, V, W, A, C, F, G, I, L, M and P;
         the amino acid at position 66 is selected from the group consisting of N, Q, S, T and Y;
         the amino acid at position 92 is selected from the group consisting of S, V, W, A C, F, G, I, L, M and P;
         the amino acid at position 93 is selected from the group consisting of S, V, W, A C, F, G, I, L, M and P;
         the amino acid at position 107 is selected from the group consisting of T, V, W, A, C, F, G, I, L, M and P;
         the amino acid at position115 is selected from the group consisting of S, V, W, A, C, F, G, I, L, M and P;
         the amino acid at position 118 is selected from the group consisting of G,
         I, L, M, P, V, W, A, C and F; and
         the amino acid at position 119 is selected from the group consisting of F, G, I, L, M, P, V, W, A and C;
      b) TCR alpha chain variable region comprising a CDR1 having the amino acid sequence of SEQ ID NO: 34, a CDR2 having the amino acid sequence of SEQ ID NO: 35 and a CDR3 having the amino acid sequence of SEQ ID NO: 36;
      Polynucleotides encoding TCR beta chains comprising
      a) TCR beta chain constant regions which are selected from the group of TCR beta constant regions which are at least about 80% identical to the amino acid sequence as set out in SEQ ID NO: 2
      wherein the amino acid at position 4 is selected from the group consisting of K, N;
      the amino acid at position 5 is selected from the group consisting of N, R, D, E, H and K;
      the amino acid at position 18 is selected from the group consisting of E, H, K, R and D;
      the amino acid at position 22 is selected from the group consisting of S, V, W, A, C, F, G, I, L, M and P;
      the amino acid at position 34 is selected from the group consisting of T, D, E, H, K, N and R;
      the amino acid at position 37 is selected from the group consisting of Y, F;
      the amino acid at position 136 is selected from the group consisting of E, V;
      the amino acid at position 157 is selected from the group consisting of T, V, W, A, C, F, G, I, L, M and P; and
      the amino acid at position 164 is selected from the group consisting of S, V, W, A, C, F, G, I, L, M and P;
      the amino acid at position 177 is selected from the group consisting of S, F;
      the amino acid at position 178 is R or deleted;
      the amino acid at position 179 is G or deleted.
      b) TCR beta chain variable region comprising a CDR1 having the amino acid sequence of SEQ ID NO: 37, a CDR2 having the amino acid sequence of SEQ ID NO: 38 and a CDR3 having the amino acid sequence of SEQ ID NO: 39.
Item 28: A library of synthetic polynucleotides encoding TCR alpha chains and TCR beta chains of a TCR capable of binding to a NY-ESO-1/LAGE-1 peptide having the sequence SLLMWITQC (SEQ ID NO: 33), comprising
   Polynucleotides encoding TCR alpha chains comprising:
      a) TCR alpha chain constant regions which are selected from the group of TCR alpha constant regions which are at least about 80% identical to the amino acid sequence as set out in SEQ ID NO: 1;
         wherein
         the amino acid at position 14 is selected from the group consisting of S, V, W, A, C, F, G, I, L, M and P;
         the amino acid at position 66 is selected from the group consisting of N, Q, S, T and Y;
         the amino acid at position 92 is selected from the group consisting of S, V, W, A C, F, G, I, L, M and P;
         the amino acid at position 93 is selected from the group consisting of S, V, W, A C, F, G, I, L, M and P;
         the amino acid at position 107 is selected from the group consisting of T, V, W, A, C, F, G, I, L, M and P;
         the amino acid at position115 is selected from the group consisting of S, V, W, A, C, F, G, I, L, M and P;
         the amino acid at position 118 is selected from the group consisting of G, I, L, M, P, V, W, A, C and F; and
         the amino acid at position 119 is selected from the group consisting of F, G, I, L, M, P, V, W, A and C;
      b) TCR alpha chain variable region comprising a CDR1 having the amino acid sequence of SEQ ID NO: 34, a CDR2 having the amino acid sequence of SEQ ID NO: 35 and a CDR3 having the amino acid sequence of SEQ ID NO:36;
   Polynucleotides encoding TCR beta chains comprising
      a) TCR beta chain constant regions which are selected from the group of TCR beta constant regions which are at least about 80% identical to the amino acid sequence as set out in SEQ ID NO: 15; wherein
      the amino acid at position 5 is selected from the group consisting of N, R, D, E, H and K;
      the amino acid at position 18 is selected from the group consisting of E, H, K, R and D;
      the amino acid at position 22 is selected from the group consisting of S, V, W, A, C, F, G, I, L, M and P;
      the amino acid at position 34 is selected from the group consisting of T, D, E, H, K, N and R;
      the amino acid at position 157 is selected from the group consisting of T, V, W, A, C, F, G, I, L, M and P; and
      the amino acid at position 164 is selected from the group consisting of S, V, W, A, C, F, G, I, L, M and P;
      or
      TCR beta chain constant regions which are selected from the group of TCR beta constant regions which are at least about 80% identical to the amino acid sequence as set out in SEQ ID NO: 16
      wherein
      the amino acid at position 5 is selected from the group consisting of N, R, D, E, H and K;
      the amino acid at position 18 is selected from the group consisting of E, H, K, R and D;
      the amino acid at position 22 is selected from the group consisting of S, V, W, A, C, F, G, I, L, M and P;
      the amino acid at position 34 is selected from the group consisting of T, D, E, H, K, N and R;
      the amino acid at position 157 is selected from the group consisting of T, V, W, A, C, F, G, I, L, M and P; and
      the amino acid at position 164 is selected from the group consisting of S, V, W, A, C, F, G, I, L, M and P;
   b) TCR beta chain variable region comprising a CDR1 having the amino acid sequence of SEQ ID NO: 37, a CDR2 having the amino acid sequence of SEQ ID NO: 38 and a CDR3 having the amino acid sequence of SEQ ID NO: 39.
Item 29: A library of synthetic polynucleotides encoding TCR alpha chains and TCR beta chains of a TCR capable of binding to a NY-ESO-1/LAGE-1 peptide having the sequence SLLMWITQC (SEQ ID NO: 33), comprising
   Polynucleotides encoding TCR alpha chains comprising:
   a) TCR alpha chain constant regions which are selected from the group of TCR alpha constant regions which are at least about 80% identical to the amino acid sequence as set out in SEQ ID NO: 1:
      wherein
      the amino acid at position 14 is selected from the group consisting of S, V, W, A, C, F, G, I, L, M and P;
      the amino acid at position 66 is selected from the group consisting of N, Q, S, T and Y;
      the amino acid at position 92 is selected from the group consisting of S, V, W, A C, F, G, I, L, M and P;
      the amino acid at position 93 is selected from the group consisting of S, V, W, A C, F, G, I, L, M and P;
      the amino acid at position 107 is selected from the group consisting of T, V, W, A, C, F, G, I, L, M and P;
      the amino acid at position115 is selected from the group consisting of S, V, W, A, C, F, G, I, L, M and P;
      the amino acid at position 118 is selected from the group consisting of G, I, L, M, P, V, W, A, C and F; and
      the amino acid at position 119 is selected from the group consisting of F, G, I, L, M, P, V, W, A and C;
   b) TCR alpha chain variable region comprising a CDR1 having the amino acid sequence of SEQ ID NO: 34, a CDR2 having the amino acid sequence of SEQ ID NO: 35 and a CDR3 having the amino acid sequence of SEQ ID NO: 36;
   Polynucleotides encoding TCR beta chains comprising
   a) TCR beta chain constant regions which are selected from the group of TCR beta constant regions which are at least about 80% identical to the amino acid sequence as set out in SEQ ID NO: 2
      wherein the amino acid at position 4 is selected from the group consisting of K, N;
      the amino acid at position 5 is selected from the group consisting of N, R, D, E, H and K;
      the amino acid at position 18 is selected from the group consisting of E, H, K, R and D;
      the amino acid at position 22 is selected from the group consisting of S, V, W, A, C, F, G, I, L, M and P;
      the amino acid at position 34 is selected from the group consisting of T, D, E, H, K, N and R;
      the amino acid at position 37 is selected from the group consisting of Y, F;
      the amino acid at position 136 is selected from the group consisting of E, V;
      the amino acid at position 157 is selected from the group consisting of T, V, W, A, C, F, G, I, L, M and P; and
      the amino acid at position 164 is selected from the group consisting of S, V, W, A, C, F, G, I, L, M and P;
      the amino acid at position 177 is selected from the group consisting of S, F;
      the amino acid at position 178 is R or deleted;
      the amino acid at position 179 is G or deleted; and
      the amino acid at position 164 is selected from the group consisting of S, V, W, A, C, F, G, I, L, M and P;
   b) TCR beta chain variable region comprising a CDR1 having the amino acid sequence of SEQ ID NO: 37, a CDR2 having the amino acid sequence of SEQ ID NO: 38 and a CDR3 having the amino acid sequence of SEQ ID NO: 39.
Item 30: Library of synthetic polynucleotides according to items 26 to 29, wherein the TCR specifically recognizes the amino acid sequence of SEQ ID NO: 33, which is presented by a molecule encoded by an HLA-A*02 gene.
Item 31: Library of synthetic polynucleotides according to items 26 to 30, wherein the library comprises polynucleotides encoding a TCR alpha chain and a TCR beta chain, each polynucleotide comprising:
   Polynucleotide encoding TCR alpha chain as defined in item 1, and
   Polynucleotide encoding TCR beta chain as defined in item 1;
   Optionally a selection marker,
   Optionally one or more ribosomal entry site or self-cleaving peptides of the 2A family.
Item 32: Library of synthetic polynucleotides according to any one of items 1 to 4, wherein the library comprises at least 1×10⁵, preferably, 1×10¹⁰, more preferably 2.1×10¹¹, such as 2.25×10¹³ unique molecules.
Item 33: A library of vectors comprising the polynucleotide library according to any one of items 1 to 5.
Item 34: The library of vectors according to item 6, wherein the vectors are retroviral vectors.
Item 35: A library of cells, comprising the library of vectors according to item 7.
Item 36: A library of cells expressing the polynucleotides defined in items 26 to 32.
Item 37: A library of polypeptides encoded by the library of synthetic polynucleotides according to items 26 to 32.
Item 38: Use of the library of items 26 to 37 for identifying a TCR receptor with enhanced TCR alpha and TRC beta chain pairing.
Item 39: A method for isolating a library derived TCR with enhanced TCR alpha and beta chain pairing, comprising the steps:
   Co-culturing the library of cells according to item 35 with antigen presenting cells presenting the HLA bound form of the target antigen of the TCR, and at least one of the steps
   Selecting cells showing elevated expression levels of the heterologous TCR compared to wild type,
   Selecting cells showing elevated activation levels compared to wild type.
Item 40: A TCR isolated from the TCR library as described in item 39.
Item 41: A library of TCR alpha chains and TCR beta chains, comprising
   TCR alpha chains comprising:
      a) TCR alpha chain constant regions which are selected from the group of TCR alpha constant regions which are at least about 80% identical to the amino acid sequence as set out in SEQ ID NO 1;
      b) TCR alpha chain variable region comprising a CDR1 having the amino acid sequence of SEQ ID NO: 34, a CDR2 having the amino acid sequence of SEQ ID NO: 35 and a CDR3 having the amino acid sequence of SEQ ID NO: 36;
   TCR beta chains comprising
      a) TCR beta chain constant regions comprising constant regions which are selected from the group of TCR beta constant regions which are at least about 80% identical to the amino acid sequence as set out in SEQ ID NO 2; or
         TCR beta chain constant regions which are selected from the group of TCR beta constant regions which are at least about 80% identical to the amino acid sequence as set out in SEQ ID NO 15;
      b) TCR beta chain variable region comprising a CDR1 having the amino acid sequence of SEQ ID NO: 37, a CDR2 having the amino acid sequence of SEQ ID NO: 38 and a CDR3 having the amino acid sequence of SEQ ID NO: 39.
Item 42: A library of TCR alpha chains and TCR beta chains, comprising
   TCR alpha chains comprising:
      a) TCR alpha chain constant regions which are selected from the group of TCR alpha constant regions which are at least about 80% identical to the amino acid sequence as set out in SEQ ID NO 1:
      wherein
      X at position 14 is selected from the group consisting of S, V, W, A, C, F, G, I, L, M and P;
      X at position 66 is selected from the group consisting of N, Q, S, T and Y;
      X at position 92 is selected from the group consisting of S, V, W, A C, F, G, I, L, M and P;
      X at position 93 is selected from the group consisting of S, V, W, A C, F, G, I, L, M and P;
      X at position 107 is selected from the group consisting of T, V, W, A, C, F, G, I, L, M and P;
      X at position115 is selected from the group consisting of S, V, W, A, C, F, G, I, L, M and P;
      X at position 118 is selected from the group consisting of G, I, L, M, P, V, W, A, C and F; and
      X at position 119 is selected from the group consisting of F, G, I, L, M, P, V, W, A and C;
   b) TCR alpha chain variable region comprising a CDR1 having the amino acid sequence of SEQ ID NO: 34, a CDR2 having the amino acid sequence of SEQ ID NO: 35 and a CDR3 having the amino acid sequence of SEQ ID NO: 36;
   TCR beta chains comprising
   a) TCR beta chain constant regions comprising constant regions which are selected from the group of TCR beta constant regions which are at least about 80% identical to the amino acid sequence as set out in SEQ ID NO: 2,
      wherein
      the amino acid at position 4 is selected from the group consisting of K, N;
      the amino acid at position 5 is selected from the group consisting of N, R, D, E, H and K;
      the amino acid at position 18 is selected from the group consisting of E, H, K, R and D;
      the amino acid at position 22 is selected from the group consisting of S, V, W, A, C, F, G, I, L, M and P;
      the amino acid at position 34 is selected from the group consisting of T, D, E, H, K, N and R;
      the amino acid at position 37 is selected from the group consisting of Y, F;
      the amino acid at position 136 is selected from the group consisting of E, V;
      the amino acid at position 157 is selected from the group consisting of T, V, W, A, C, F, G, I, L, M and P; and
      the amino acid at position 164 is selected from the group consisting of S, V, W, A, C, F, G, I, L, M and P;
      the amino acid at position 177 is selected from the group consisting of S, F;
      the amino acid at position 178 is R or deleted;
      the amino acid at position 179 is G or deleted.
   b) TCR beta chain variable region comprising a CDR1 having the amino acid sequence of SEQ ID NO: 37, a CDR2 having the amino acid sequence of SEQ ID NO: 38 and a CDR3 having the amino acid sequence of SEQ ID NO: 39.
Item 43: A method of preparing a library of synthetic polynucleotides encoding a plurality of encoding TCR alpha chain constant regions and TCR beta chain constant regions, the method comprising:
   a) Providing the sequences of the polynucleotides encoding the TCR alpha chains and TCR beta chains as defined in items 26 to 32;
   b) Assembling the synthetic polynucleotide to produce a library of synthetic polynucleotides.
Item 44: A kit comprising the library as defined in items 36 to 37, 41 or 42.
Item 45: The nucleotide sequences of the synthetic polynucleotides defined in items 26 to 32 in computer readable format.
Item 46: The amino acid sequences defined in items 26 to 32 in computer readable format.
Item 47: TCR capable of binding to a NY-ESO-1/LAGE-1 peptide having the sequence SLLMWITQC (SEQ ID NO: 33), wherein the TCR comprises:
   a TCR alpha chain comprising
a) TCR alpha chain variable region comprising a CDR1 having the amino acid sequence of SEQ ID NO: 34, a CDR2 having the amino acid sequence of SEQ ID NO: 35 and a CDR3 having the amino acid sequence of SEQ ID NO: 36;
b) a constant TCR alpha region comprising the sequence selected from the group of constant TCR alpha regions represented by the sequence as set in SEQ ID NO 1:
a TCR beta chain comprising
a) TCR beta chain variable region comprising a CDR1 having the amino acid sequence of SEQ ID NO: 37, a CDR2 having the amino acid sequence of SEQ ID NO: 38 and a CDR3 having the amino acid sequence of SEQ ID NO: 39;
b) a constant TCR beta region comprising the sequence selected from the group of constant TCR beta regions represented by the sequence as set in SEQ ID NO: 15, or
   a constant TCR beta region comprising the sequence selected from the group of constant TCR beta regions represented by the sequence as set in SEQ ID NO: 16.
Item 48: TCR according to item 47, wherein the TCR shows elevated expression levels of the heterologous TCR compared to wild type.
Item 49: TCR according to items 47 and 48, wherein the TCR shows elevated activation levels compared to wild type.
Item 50: TCR according to items 47 to 49, wherein at in least one of X01 to X14 the amino acid is not the amino acid which occurs naturally in wild type at the respective position.
Item 51: The TCR according to items 47 to 50, wherein in at least two, at least three, at least four, at least five, at least 6, at least 7, at least 8, at least 9, at least 10, at least 11, at least 12, at least 13, such as in 14 positions of X01 to X14, the amino acid is not the amino acid which occurs naturally in wild type at the respective position.
Item 52. TCR capable of binding to a NY-ESO-1/LAGE-1 peptide having the sequence SLLMWITQC (SEQ ID NO: 33), wherein the TCR comprises:
   a TCR alpha chain comprising
   a) a variable TCR alpha chain region comprising a CDR1 having the amino acid sequence of SEQ ID NO: 34, a CDR2 having the amino acid sequence of SEQ ID NO: 35 and a CDR3 having the amino acid sequence of SEQ ID NO: 36;
   b) A constant TCR alpha region comprising the sequence SEQ ID NO: 12
   A TCR beta chain comprising
      a) a variable TCR beta chain region comprising a CDR1 having the amino acid sequence of SEQ ID NO: 37, a CDR2 having the amino acid sequence of SEQ ID NO: 38 and a CDR3 having the amino acid sequence of SEQ ID NO: 39;
      b) a constant TCR beta region comprising the sequence SEQ ID NO: 13;
Item 53. TCR according to items 47 to 52, wherein the TCR specifically recognizes the amino acid sequence of SEQ ID NO: 33, which is presented by a molecule encoded by an HLA-A*02 gene.
Item 54. TCR according to any one of items 47 to 53, wherein the TCR comprises a) a variable TCR α region having an amino acid sequence which is at least 80% identical to SEQ ID NO: 40 and b) a variable TCR β region having an amino acid sequence which is at least 80% identical to SEQ ID NO: 41.
Item 55: TCR according to any one of items 47 to 54, wherein the TCR comprises
   a) a variable TCR α region having the amino acid sequence of SEQ ID NO: 40 and
   b) a variable TCR β region having the amino acid sequence of SEQ ID NO: 41.
Item 56: Nucleic acid encoding a TCR according to any one of items 47 to 55.
Item 57: Vector comprising the nucleic acid of item 56.
Item 58: Vector according to item 57, wherein the vector is an expression vector.
Item 59: Vector according to item 57 or 58, wherein the vector is a retroviral vector.
Item 60: Vector according to item 57 or 59, wherein the vector is a lentiviral vector.
Item 61: Cell expressing the TCR according to items 47 to 55, the nucleic acid according to item 56, the vector according to items 57 to 60.
Item 62: Cell according to item 61, wherein the cell is isolated or non-naturally occurring.
Item 63: Cell according to items 61 or 62, wherein the cell comprises the nucleic acid according to item 56 or the vector according to items 57 to 60.
Item 64: Cell according to items 61 to 63, wherein the cell comprises:
a) an expression vector which comprises at least one nucleic acid as embodied in item 56, or
b) a first expression vector which comprises a nucleic acid encoding the alpha chain of the TCR as embodied in any one of the items 47 to 55, and a second expression vector which comprises a nucleic acid encoding the beta chain of a TCR as embodied in any one of the items 47 to 55.
Item 65: Cell according to any one of items 61 to 64, wherein the cell is a peripheral blood lymphocyte (PBL) or a peripheral blood mononuclear cell (PBMC).
Item 66: Cell according to any one of items 61 to 65, wherein the cell is a T cell.
Item 67: Pharmaceutical composition comprising the TCR according to items 47 to 55, the nucleic acid according to item 56, the vector according to items 57 to 60, the cell according to any one of items 61 to 66.
Item 68: Pharmaceutical composition according to item 67 wherein the pharmaceutical composition comprises at least one pharmaceutically acceptable carrier.
Item 69: The TCR according to items 47 to 55, the nucleic acid according to item 56, the vector according to items 57 to 60, the cell according to any one of items 61 to 66 for use as a medicament.
Item 70: The TCR according to items 47 to 55, the nucleic acid according to item 56, the vector according to items 57 to 60, the cell according to any one of items 61 to 66 for use in the treatment of cancer.
Item 71: The TCR, the polypeptide, the multivalent TCR complex, the nucleic acid, the vector or the cell for use according to item 70, wherein the cancer is a hematological cancer or a solid tumor.

1. A library of synthetic polynucleotides encoding TCR alpha chain constant regions and TCR beta chain constant regions, wherein the library comprises:
Polynucleotides encoding TCR alpha chain constant regions which are selected from the group of TCR alpha constant regions which are at least about 80% identical to the amino acid sequence as set out in SEQ ID NO: 1,
wherein
the amino acid at position 14 is selected from the group consisting of S, V, W, A, C, F, G, I, L, M and P;
the amino acid at position 66 is selected from the group consisting of N, Q, S, T and Y;
the amino acid at position 92 is selected from the group consisting of S, V, W, A C, F, G, I, L, M and P;
the amino acid at position 93 is selected from the group consisting of S, V, W, A C, F, G, I, L, M and P;
the amino acid at position 107 is selected from the group consisting of T, V, W, A, C, F, G, I, L, M and P;
the amino acid at position115 is selected from the group consisting of S, V, W, A, C, F, G, I, L, M and P;
the amino acid at position 118 is selected from the group consisting of G, I, L, M, P, V, W, A, C and F; and
the amino acid at position 119 is selected from the group consisting of F, G, I, L, M, P, V, W, A and C;
Polynucleotides encoding TCR beta chain constant regions which are selected from the group of TCR beta constant regions which are at least about 80% identical to the amino acid sequence as set out in SEQ ID NO: 2
wherein
the amino acid at position 4 is selected from the group consisting of K, N;
the amino acid at position 5 is selected from the group consisting of N, R, D, E, H and K;
the amino acid at position 18 is selected from the group consisting of E, H, K, R and D;
the amino acid at position 22 is selected from the group consisting of S, V, W, A, C, F, G, I, L, M and P;
the amino acid at position 34 is selected from the group consisting of T, D, E, H, K, N and R;
the amino acid at position 37 is selected from the group consisting of Y, F;
the amino acid at position 136 is selected from the group consisting of E, V;
the amino acid at position 157 is selected from the group consisting of T, V, W, A, C, F, G, I, L, M and P; and
the amino acid at position 164 is selected from the group consisting of S, V, W, A, C, F, G, I, L, M and P;
the amino acid at position 177 is selected from the group consisting of S, F;
the amino acid at position 178 is R or deleted;
the amino acid at position 179 is G or deleted.

2. A library of synthetic polynucleotides encoding TCR alpha chains and TCR beta chains, comprising
Polynucleotides encoding TCR alpha chains comprising constant regions which are selected from the group of TCR alpha constant regions which are at least about 80% identical to the amino acid sequence as set out in SEQ ID NO: 1;
wherein
the amino acid at position 14 is selected from the group consisting of S, V, W, A, C, F, G, I, L, M and P;
the amino acid at position 66 is selected from the group consisting of N, Q, S, T and Y;
the amino acid at position 92 is selected from the group consisting of S, V, W, A C, F, G, I, L, M and P;
the amino acid at position 93 is selected from the group consisting of S, V, W, A C, F, G, I, L, M and P;
the amino acid at position 107 is selected from the group consisting of T, V, W, A, C, F, G, I, L, M and P;
the amino acid at position115 is selected from the group consisting of S, V, W, A, C, F, G, I, L, M and P;
the amino acid at position 118 is selected from the group consisting of G, I, L, M, P, V, W, A, C and F; and
the amino acid at position 119 is selected from the group consisting of F, G, I, L, M, P, V, W, A and C;
Polynucleotides encoding TCR beta chains comprising constant regions which are selected from the group of TCR beta constant regions which are at least about 80% identical to the amino acid sequence as set out in SEQ ID NO: 2
wherein
the amino acid at position 4 is selected from the group consisting of K, N;
the amino acid at position 5 is selected from the group consisting of N, R, D, E, H and K;
the amino acid at position 18 is selected from the group consisting of E, H, K, R and D;
the amino acid at position 22 is selected from the group consisting of S, V, W, A, C, F, G, I, L, M and P;
the amino acid at position 34 is selected from the group consisting of T, D, E, H, K, N and R;
the amino acid at position 37 is selected from the group consisting of Y, F;
the amino acid at position 136 is selected from the group consisting of E, V;
the amino acid at position 157 is selected from the group consisting of T, V, W, A, C, F, G, I, L, M and P; and
the amino acid at position 164 is selected from the group consisting of S, V, W, A, C, F, G, I, L, M and P;
the amino acid at position 177 is selected from the group consisting of S, F;
the amino acid at position 178 is R or deleted;
the amino acid at position 179 is G or deleted.

3. Library of synthetic polynucleotides according to any one of claim 1 and claim 2, wherein the library comprises polynucleotides encoding a TCR alpha chain and a TCR beta chain, each polynucleotide comprising:
Polynucleotide encoding TCR alpha chains as defined in claim 2; and
Polynucleotide encoding TCR beta chains as defined in claim 2;
Optionally a selection marker
Optionally one or more ribosomal entry site or self-cleaving peptides of the 2A family.

4. Library of synthetic polynucleotides according to claim 3, wherein the selection marker is a fluorescent tag.

5. Library of synthetic polynucleotides according to claim 4, wherein the fluorescent tag is MTagBFP.

6. Library of synthetic polynucleotides according to of claims 1 to 5, wherein the library comprises at least 1×10⁵, preferably, 1×10¹⁰, more preferably 2.1×10¹¹, such as 2.25×10¹³ unique molecules.

7. A library of vectors comprising the polynucleotide library according to any one of claims 1 to 6.

8. The library of vectors according to claim 7, wherein the vectors are retroviral vectors.

9. A library of cells, comprising the library of vectors according to claim 8.

10. A library of cells expressing the polynucleotides defined in claims 1 to 6.

11. A library of polypeptides encoded by the library of synthetic polynucleotides according to claims 1 to 6.

12. Use of the library of claims 1 to 11 for identifying a TCR receptor with enhanced TCR alpha and TCR beta chain pairing.

13. A method for isolating a TCR with enhanced TCR alpha and beta chain pairing, comprising the steps:
Co-culturing the library of cells according to claims 9 to 11 with antigen presenting cells presenting the HLA bound form of the target antigen of the TCR
Selecting cells showing elevated expression levels of the heterologous TCR compared to wild type
Selecting cells showing elevated activation levels compared to wild type.

14. A TCR isolated from the TCR library as described in claim 13.

15. A library of TCR alpha chains and TCR beta chains, comprising
TCR alpha chains comprising constant regions which are selected from the group of TCR alpha constant regions which are at least about 80% identical to the amino acid sequence as set out in SEQ ID NO: 1;
wherein
the amino acid at position 14 is selected from the group consisting of S, V, W, A, C, F, G, I, L, M and P;
the amino acid at position 66 is selected from the group consisting of N, Q, S, T and Y;
the amino acid at position 92 is selected from the group consisting of S, V, W, A C, F, G, I, L, M and P;
the amino acid at position 93 is selected from the group consisting of S, V, W, A C, F, G, I, L, M and P;
the amino acid at position 107 is selected from the group consisting of T, V, W, A, C, F, G, I, L, M and P;
the amino acid at position115 is selected from the group consisting of S, V, W, A, C, F, G, I, L, M and P;
the amino acid at position 118 is selected from the group consisting of G, I, L, M, P, V, W, A, C and F; and
the amino acid at position 119 is selected from the group consisting of F, G, I, L, M, P, V, W, A and C;
TCR beta chains comprising constant regions which are selected from the group of TCR beta constant regions which are at least about 80% identical to the amino acid sequence as set out in SEQ ID NO: 2
wherein
the amino acid at position 4 is selected from the group consisting of K, N;
the amino acid at position 5 is selected from the group consisting of N, R, D, E, H and K;
the amino acid at position 18 is selected from the group consisting of E, H, K, R and D;
the amino acid at position 22 is selected from the group consisting of S, V, W, A, C, F, G, I, L, M and P;
the amino acid at position 34 is selected from the group consisting of T, D, E, H, K, N and R;
the amino acid at position 37 is selected from the group consisting of Y, F;
the amino acid at position 136 is selected from the group consisting of E, V;
the amino acid at position 157 is selected from the group consisting of T, V, W, A, C, F, G, I, L, M and P; and
the amino acid at position 164 is selected from the group consisting of S, V, W, A, C, F, G, I, L, M and P;
the amino acid at position 177 is selected from the group consisting of S, F;
the amino acid at position 178 is R or deleted;
the amino acid at position 179 is G or deleted.

16. A method of preparing a library of synthetic polynucleotides encoding a plurality of TCR alpha chain constant regions and TCR beta chain constant regions, the method comprising:
Providing sequences of polynucleotides
(iii) encoding TCR alpha chain constant regions and TCR beta chain constant regions as defined in claim 1; or
(iv) encoding the TCR alpha chain and TCR beta chain as defined in claim 2;
Assembling the synthetic polynucleotide sequences to produce a library of synthetic polynucleotides.

17. A kit comprising the library as defined in claims 1 to 11.

18. The nucleotide sequences of the synthetic polynucleotides defined in claims 1 to 11 in computer readable format.

19. The amino acid sequences defined in claims 1 to 11 in computer readable format.

20. A TCR, comprising
A TCR alpha chain comprising constant region which is at least about 80% identical to the amino acid sequence as set out in SEQ ID NO: 1;
TCR beta chains comprising a constant regions which is at least about 80% identical to the amino acid sequence as set out in SEQ ID NO: 2.
